(19) United States
(12) Patent Application Publication
(10) Pub. No.: US 2025/0367330 A1
(43) Pub. Date: Dec. 4, 2025

(54) OPTICAL CLEARING ENABLED BY THE KRAMERS-KRONIG RELATION

(71) Applicant: The Board of Trustees of the Leland Stanford Junior University, Stanford, CA (US)

(72) Inventors: Guosong Hong, Palo Alto, CA (US); Zihao Ou, Palo Alto, CA (US); Nicholas Joseph Rommelfanger, Stanford, CA (US); Shan Jiang, Redwood City, CA (US); Mark L Brongersma, Stanford, CA (US); Yi-Shiou Duh, New Taipei City (TW); Su Zhao, Santa Clara, CA (US); Kenneth Brinson, Stanford, CA (US)

(21) Appl. No.: 18/707,919

(22) PCT Filed: Dec. 19, 2022
(86) PCT No.: PCT/US2022/081931

§ 371 Date: May 7, 2024

Related U.S. Application Data
(60) Provisional application No. 63/265,726, filed on Dec. 20, 2021.

(51) Int. Cl. A61K 49/00

(52) U.S. Cl. CPC A61K 49/00

(57) ABSTRACT

In one aspect, the disclosure relates to compositions and use thereof including an absorbing substance that absorbs light at specific wavelengths, thus allowing the refractive index of the medium at neighboring wavelengths to be modulated accordingly. This modulation allows reduction of the refractive index mismatch between separate phases with distinct refractive indices, thus mitigating scattering. The compositions are soluble in water and have high absorptivity, thus significantly increasing the refractive index at desired wavelengths. In one aspect, these materials have been proved safe in oral administration. In another aspect, provided herein is a protocol that enables diffusion of these substances into the aqueous phase of biological tissues, including in living organisms, thereby achieving increased transmission in muscles and allowing clear visualization of deep bones, vessels, and nerves that are otherwise invisible in the body.

### CROSS REFERENCE TO RELATED APPLICATIONS

This application claims the benefit of U.S. Provisional Application No. 63/265,726, filed on Dec. 20, 2021, which is incorporated herein by reference in its entirety.

### STATEMENT REGARDING FEDERALLY SPONSORED RESEARCH OR DEVELOPMENT

This invention was made with government support under contract number 2045120 awarded by the National Science Foundation. The government has certain rights in the invention.

### BACKGROUND

The complex structure of biological matter endows it with a tremendous diversity of functions while also giving rise to unwanted light scattering that renders it opaque. The desire to see inside biomaterials and explore the basic processes of life has motivated extensive research on optical tissue clearing. Existing clearing methods are largely aimed at reducing the scattering associated with the spatial variations in the refractive index by dehydration and hyperhydration. However, these processes remove water and lipids, thus precluding their use in live organisms.

Biomedical imaging plays a central role in clinical analysis and medical intervention while allowing for non-invasive studies of complex biological processes. However, optical imaging of biological tissues is fundamentally limited by scattering and absorption of light. In most tissues, the scattering coefficient is 10-1000 times larger than the absorption coefficient; thus, scattering processes can severely limit the imaging depth and spatial resolution in conventional microscopy. For this reason, the ability to achieve significant reductions in light scattering holds promise for transforming brightfield, fluorescence, nonlinear, and super-resolution imaging techniques.

Light scattering in tissue originates from the optical contrast between low refractive index (RI) aqueous-based components (e.g., the interstitial fluid and cytosol) and high RI lipid-based components (e.g., the plasma membrane, myelin, and myofibrils) as illustrated in FIG. 2A. Existing methods to reduce optical contrast usually replace water with high-RI chemicals or remove lipids to yield an all-aqueous environment. Despite their success, these approaches are seldom employed in live tissues as they involve the use of toxic substances (e.g., tetrahydrofuran and acrylamide) and removal of molecules vital to sustaining life (e.g., water and lipids).

Despite advances in optical imaging research, there is still a scarcity of compounds and methods that are nontoxic and biocompatible and also effective at reducing refractive index differences between and among different tissue types in an organ to be imaged, in addition to being useful in non-living systems where optical transparency is desired. These needs and other needs are satisfied by the present disclosure.

### SUMMARY

In accordance with the purpose(s) of the present disclosure, as embodied and broadly described herein, the disclosure, in one aspect, relates to compositions and use thereof including an absorbing substance that absorbs light at specific wavelengths, thus allowing the refractive index of the medium at neighboring wavelengths to be modulated accordingly. This modulation allows reduction of the refractive index mismatch between separate phases with distinct refractive indices, thus mitigating scattering. The compositions are soluble in water and have high absorptivity, thus significantly increasing the refractive index at desired wavelengths. In one aspect, these materials have been proved safe in oral administration. In another aspect, provided herein is a protocol that enables diffusion of these substances into the aqueous phase of biological tissues, including in living organisms, thereby achieving increased transmission in muscles and allowing clear visualization of deep bones, vessels, and nerves that are otherwise invisible in the body (FIG. 1).

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows a schematic illustrating the disclosed method for in vivo optical clearing.

FIGS. 2A-2I show principles of the disclosed method. (FIG. 2A) Schematic illustrating the n′ mismatch between the scatterers (e.g., lipids, collagen fibers, and the nucleus) and the aqueous background (e.g., interstitial fluid and cytoplasm) in biological tissue. This mismatch leads to distortion of the scattered wavefront. (FIG. 2B) Schematic illustrating the reduced index mismatch between the scatterers and aqueous background in biological tissue after raising the index of its aqueous components via optical clearing via the Kramers-Kronig relation (herein referred to for convenience as “K-Klear”). (FIG. 2C) Numerical simulation showing the modulation of the real index of water (n′, purple solid line) by introducing an absorbing molecule with a peak in n″ (gray solid line) centered at 428 nm. The n′ of pure water and high-RI cellular components (e.g., lipids and collagen fibers) are also included. (FIGS. 2D-2E) Imaginary part n″ (FIG. 2D) and real part n′ (FIG. 2E) of the RI calculated from a Lorentz model with oscillating frequency ω₀ at 100 nm and 400 nm, respectively. (FIGS. 2F-2G) Molar absorption a (FIG. 2F) and molar n′ change β (FIG. 2G) of glycerol and Dye-4 (tartrazine) dissolved in water, respectively. (FIGS. 2H-2I) Wavelength dependence of maximum molar absorption a (FIG. 2F) and maximum molar n′change β (FIG. 2G) for 23 chemicals listed in Table 1.

FIGS. 3A-3E show screening of absorbing molecules to enable the K-Klear method. (FIG. 3A) The relation between average molar absorption a and average molar n′ change β in the wavelength range between 500 to 700 nm for different molecules. (FIG. 3B) The ratio between the molar n′ change and molar absorption (β/α) for different absorbing molecules. The “white window” indicates a potential clearing spectrum with sufficient Δn′ and minimal absorption. (FIGS. 3C-3D) Imaginary part n″ (FIG. 3C) and real part n′ (FIG. 3D) of the RI of Dye-4 solutions at different concentrations measured from ellipsometry. (FIG. 3E) Normal-incidence light transmittance T of Dye-4 solutions with a fixed optical path length of 1 mm measured at different concentrations, showing the transmission window beyond 600 nm despite strong absorption below 500 nm.

FIGS. 4A-4G show a demonstration of K-Klear in scattering phantoms. (FIGS. 4A-4B) Schematics illustrating the scatter phantom composed of monodispersed silica particles distributed in an aqueous medium before (FIG. 4A) and after (FIG. 4B) K-Klear. (FIG. 4C) Numerical simulation showing a plane wave traveling through a matrix comprising nanoparticles in an aqueous background with different levels of n′ mismatch indicated above each graph. Scale bar: 2 μm. (FIG. 4D) Photographs of scattering phantoms composed of agarose hydrogels containing different concentrations of Dye-4 while keeping the concentration of silica particles the same. Scale bars: 5 mm. (FIG. 4E) Normal-incidence light transmittance T spectra of the scattering phantom containing different concentrations of Dye-4. (FIG. 4F) Spectra of transmission enhancement factor, which is defined as the transmittance ratio between the phantom containing a specific concentration of Dye-4 and that containing 0 M Dye-4. (FIG. 4G) The ratio of attenuation coefficient between scattering phantoms containing different concentrations of Dye-4 and that containing 0 M Dye-4. Plots in FIGS. 4E-4G follow the same color scheme as shown in FIG. 4E.

FIGS. 5A-5M show imaging a resolution target through scattering phantoms containing different Dye-4 concentrations. (FIGS. 5A-5B) Schematics showing the clearing effect through modulating the background RI in the scattering phantom via K-Klear, thus yielding higher resolution for imaging the USAF resolution target. (FIGS. 5C-5D) Intensity-normalized low-magnification (FIG. 5C) and high-magnification (FIG. 5D) images of the USAF resolution target through 1-mm scattering phantoms comprising different dye concentrations and imaged at different wavelengths. (FIGS. 5E-5H) Modulation transfer function (MTF) of the imaging system with scattering phantoms at 525 nm (FIG. 5E), 600 nm (FIG. 5F), 680 nm (FIG. 5G), and 785 nm (FIG. 5H) wavelengths. The solid lines in FIGS. 5E-5H share the same color scheme as denoted in FIG. 5C. (FIG. 5I) Dependence of MTF at a fixed spatial frequency of 101.6 lp mm⁻¹ on the dye concentrations at different wavelengths. (FIG. 5J) Dependence of MTF at a fixed spatial frequency of 101.6 lp mm⁻¹ on the n′ difference between the background and particles in the scattering phantom. Symbols denote different wavelengths at which MTF was measured by following the same symbol convention in FIG. 5I. (FIGS. 5K-5L) Simulated electric field E distribution of a 600-nm Gaussian beam launched from the left and propagating for 1 mm in a medium comprising silica scatterers (white dots) in water (FIG. 5K) and in an aqueous solution of 0.62 M Dye-4 (FIG. 5L). The divergent beam is refocused by an imaginary lens placed 100 μm after light exits the scattering medium. The white dashed line indicates the plane where the propagating wave is refocused. Scale bars denote 200 μm. (FIG. 5M) Electric field strength at the focal plane of light (along white dashed lines in FIGS. 5K-5L).

FIGS. 6A-6K show ex vivo K-Klear in chicken breast tissue. (FIG. 6A) Schematic illustrating the 3-step tissue clearing procedure. (FIG. 6B) Photographs illustrating the transmittance change in chicken breast tissue after each step. Scale bars: 1 cm. (FIG. 6C) Transmittance spectra and (FIG. 6D) transmission enhancement factor of the muscle tissue after immersion in different concentrations of Dye-4 in Step-3. (FIG. 6E) Ratio of the attenuation coefficient of tissue immersed in Dye-4 solutions of different concentrations over that of the original, uncleared tissue. Lines in c-e share the same color scheme denoted in FIG. 6C. (FIG. 6F) Temporal evolution of transmittance through a chicken breast tissue sample directly immersed in a 0.62 M solution of Dye-4. The white dashed line indicates 700 nm. (FIG. 6G) Temporal evolution of transmittance and attenuation coefficient ratio at 700 nm for the tissue immersed in a 0.62 M dye solution. (FIGS. 6H-6K) Simulated spatial distribution of Dye-4 concentration (FIG. 6H), modulated n′ (FIG. 6I), scattering coefficient (FIG. 6J), and incident light trajectory at 700 nm (FIG. 6K) in a 1.8-mm thick muscle tissue (matching the thickness of samples in FIGS. 6B and 6F) before (top) and after (bottom) K-Klear. The coordinates in FIGS. 6H-6K follow the same convention of labeled axes as in FIG. 6A. Scale bars in FIGS. 6H-6K: 1 mm.

FIGS. 7A-7H show in vivo optical clearing in live mice via the K-Klear method. (FIG. 7A) Schematic illustrating the procedure of in vivo optical clearing via topical application of a hydrogel containing Dye-4 molecules on the scalp. (FIG. 7B) Photograph of the mouse head with depilated but intact scalp before optical clearing. (FIGS. 7C-7D) Laser speckle contrast images of the mouse head before (FIG. 7C) and after (FIG. 7D) topical application of the Dye-4 gel. The inferior cerebral vein, superior sagittal sinus, and transverse sinus are labeled 1, 2, and 3, respectively. Scale bars in FIGS. 7B-7D: 5 mm. (FIG. 7E) Schematic illustrating the procedure for in vivo optical clearing via topical application of a hydrogel containing Dye-4 molecules for noninvasive muscle sarcomere imaging. (FIGS. 7F-7H) Images based on the SHG signals after 1040 nm excitation collected from the mouse tibialis anterior muscle in the mouse hind limb before (FIG. 7F) and after (FIGS. 7G-7H) optical clearing. Scale bars in FIGS. 7F-7H: 50 μm.

FIGS. 8A-8C show a numerical simulation of RI modulation following the Kramers-Kronig relation. (FIG. 8A) Hypothetical n″ spectra with a Gaussian absorption peak centered at 428 nm and standard deviation of 40 nm. Each spectrum has a different peak absorption value as labeled. (FIG. 8B) The calculated n′modulation (Δn′, the deviation of n′ from that in the absence of the hypothetical absorption peak in FIG. 8A) corresponding to different absorption spectra in (FIG. 8A) using the Kramers-Kronig relation. (FIG. 8C) Linear correlation between the maximum modulation of n′ at 480 nm and peak magnitude of n″ at 428 nm.

FIGS. 9A-9E show ‘and n″ spectra of several representative dyes in Table 1. Real part (n’, left axis, solid lines) and imaginary part (n″, right axis, dashed lines) of the RI of (FIG. 9A) Dye-5 at 1 M, (FIG. 9B) Dye-7 at 1 M, (FIG. 9C) Dye-17 at 0.3 M, and (FIG. 9D) Dye-16 at 0.5 M. (FIG. 9E) The absorbance spectrum of different dyes measured through a 1-mm path length.

FIGS. 10A-10D show changing the n′ of Dye-4 solutions at different concentrations. (FIG. 10A) Chemical structure of Dye-4. (FIG. 10B) Photograph of Dye-4 hydrogels at different concentrations. (FIG. 10C) n′ of Dye-4 solutions of different concentrations measured by ellipsometry. (FIG. 10D) Dependence of n′ on molar concentrations at 500 nm, 600 nm, 700 nm, and 800 nm. Dashed lines represent linear fitting of each curve, from which the slopes are extracted to represent the molar n′ change, β, at different wavelengths in Table 2.

FIGS. 11A-11C show absorption of Dye-4 solutions at different concentrations. (FIG. 11A) Absorption spectra of Dye-4 solutions at different concentrations with a 1-mm path length. (FIG. 11B) Absorption at 428 nm plotted as a function of dye concentration. An extinction coefficient of 2.04±0.02×104 M⁻¹ cm⁻¹ is extracted from the slope of linear fitting. (FIG. 11C) Dependence of absorption on molar concentrations at 600 nm, 700 nm, and 800 nm. The smallest measurable absorbance value on the UV-Vis-NIR spectrometer is ca. 0.04, which sets the lower bound of data points in FIG. 11C.

FIG. 12 shows attenuation coefficients μₜ of the scatter phantoms shown in FIGS. 4A-4G prepared with different concentrations of Dye-4 solutions. Note that μₜ is dominated by the scattering coefficient, μₛ, for the Dye-4 concentration of 0 M.

FIG. 13 shows a numerical comparison between the theory and experimental results of the normalized attenuation coefficient as a function of n′ matching. The dashed curve represents the theoretical prediction of the scattering coefficient vs. background n′ in a scattering medium composed of monodisperse spheres. The solid curve is the experimental results at 600 nm extracted from phantoms made with 1 μm silica nanoparticles, respectively. The gray vertical line indicates the n′ of silica spheres in the scattering phantom. Due to minimal absorption of Dye-4 at 600 nm, the attenuation coefficient t is mainly contributed by the scattering coefficient s.

FIGS. 14A-14C show characterizations of 1 μm silica particles used in the scattering phantom. (FIG. 14A) Scanning electron micrograph of the silica particles. Scale bar: 5 μm. (FIG. 14B) Size distribution of the silica particles from SEM images (1013±33 nm, n=50). (FIG. 14C) Size distribution of the silica particles in a 1 mg mL⁻¹ suspension measured via dynamic light scattering.

FIGS. 15A-15E show characterizations of 1-mm thick scattering phantoms containing 1-μm silica particles and different Dye-4 concentrations. (FIG. 15A) Photographs of scattering phantoms taken with a back-illuminating LED light panel. Scale bar: 5 mm. (FIG. 15B) Transmittance spectra of the scattering phantoms. (FIG. 15C) Spectra of transmission enhancement factor, which is calculated as the ratio of the transmittance of a phantom containing a certain dye concentration over that containing 0 M of the dye. (FIG. 15D) Attenuation coefficients of the scattering phantoms. (FIG. 15E) The ratio of attenuation coefficient of the scattering phantom containing a certain dye concentration over that containing 0 M of the dye. Plots in FIGS. 15B-15E follow the same color scheme as shown in FIG. 15B.

FIG. 16 shows a schematic illustrating the imaging setup for measuring the spatial frequency resolution with a 1951 USAF resolution target.

FIGS. 17A-17C show attenuation coefficients of original chicken breast tissues. (FIG. 17A) Attenuation spectra of 10 pieces of original chicken breast tissues prepared with different tissue thicknesses. (FIG. 17B) Dependence of attenuation at 700 nm on chicken breast tissues thickness. (FIG. 17C) Attenuation coefficient of chicken breast tissues calculated from the 10 pieces of chicken breast tissues. The gray shadow indicates the standard deviation.

FIG. 18 shows transmittance spectra of chicken breast tissues before clearing. Note that all samples had comparable thicknesses to begin with, as evidenced by similar transmittance of these tissues. These curves are coded with the same colors as their corresponding samples in FIG. 6C.

FIGS. 19A-19C show extracting the diffusion coefficient of Dye-4 in the chicken breast tissue from the temporal evolution of transmittance. (FIG. 19A) Temporal evolution of transmittance T at different wavelengths of a chicken breast tissue immersed in a 0.62 M solution of Dye-4. (FIG. 19B) Temporal evolution of normalized T at different wavelengths. T is normalized between 0 and 1 against minimum and maximum T for each wavelength. (FIG. 19C) Temporal evolution of normalized T averaged over measurements at all wavelengths in FIG. 19B, enabling the extraction of the diffusion constant by fitting the normalized T to

$$1 - {e}^{- \frac{t}{τ}} .$$

Here, τ is measured to be 85.0±1.2 min. This tissue sample has a thickness of d=1.82 mm; therefore, the diffusion coefficient D of Dye-4 therein can be calculated as

$$D = \frac{{d}^{2}}{{π}^{2} τ} = 6.6 \times 1 {0}^{- 7} {cm}^{2} {s}^{- 1} .$$

FIGS. 20A-20D show in vivo K-Klear of the mouse scalp after topical application of the Dye-4 gel. (FIG. 20A) Brightfield photograph of the mouse head with the depilated but intact scalp before K-Klear. (FIGS. 20B-20D) Laser speckle contrast images of the mouse head before (FIG. 20B), after topical application of the Dye-4 gel (FIG. 20C), and after scalp removal (FIG. 20D). The same colormap is used for FIGS. 20B-20D. Scale bars: 5 mm.

FIGS. 21A-21L show line cross-sectional analysis of cerebral vessels in transdermal in vivo brain imaging. (FIGS. 21A-21B) Representative laser speckle contrast images of the mouse head in FIGS. 20A-20D through the scalp before (FIG. 21A) and after (FIG. 21B) K-Klear. (FIGS. 21C-21F) Four representative cross-sectional intensity profiles along white lines in FIGS. 21A-21B. All intensity profiles are normalized between the maximum and minimum values in both curves of each plot. (FIGS. 21G-21H) Representative laser speckle contrast images of the mouse head in FIGS. 7A-7H through the scalp before (FIG. 21G) and after (FIG. 21H) K-Klear. (FIGS. 21I-21L) Four representative cross-sectional intensity profiles along white lines in FIGS. 21G-21H. Scale bars in FIGS. 21A-21B and 21G-21H: 5 mm.

FIGS. 22A-22C show sarcomere length measurements in the live mouse hindlimb with K-Klear. (FIG. 22A) Second harmonic generation image of the mouse muscle in FIG. 7H with three representative profiles overlaid. (FIG. 22B) Cross-sectional intensity profiles along white lines in (FIG. 22A). (FIG. 22C) Peak-to-peak distance distribution of three intensity profiles respectively highlighting the sarcomere signatures. Each group includes 10 data points.

FIGS. 23A-23F show in vivo SHG images of mouse muscle fibers captured before and after K-Klear. (FIGS. 23A-23C) Original SHG images before (FIG. 23A) and after (FIGS. 23B-23C) optical clearing. (FIGS. 23D-23F) Band-pass filtered images of the original SHG images for highlighting sarcomere features by following a previous protocol. Scale bars: 50 μm.

FIG. 24 shows osmolality values of Dye-4 solution at different concentrations. Dashed line is the linear fitting of the data.

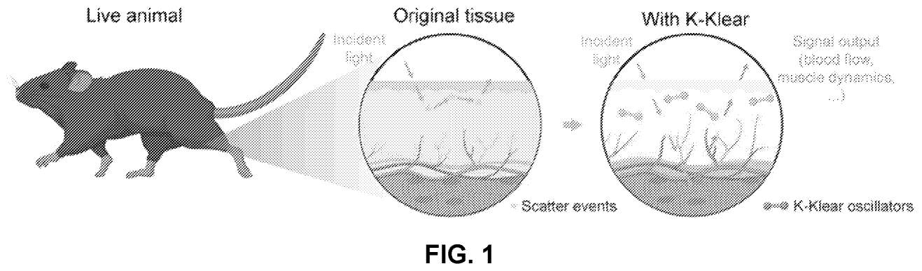

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Disclosed herein is a strategy for tissue clearing that enables effective modulation of the refractive index of a specific tissue component by tuning its absorbance. This approach leverages the Kramers-Kronig (K-K) relation, which causally connects the real and imaginary components of the refractive index of a material. Specifically, by increasing the absorbance of the aqueous component in the tissue, its RI (originally 1.35˜1.37) is modulated to match that of lipids and myofibrils (1.39˜1.52) to reduce scattering at their interfaces. Since RI modulation occurs at a different wavelength than the absorption peak, this approach yields the counterintuitive effect that greater absorption leads to an increase in transmission. After screening >20 candidates, tartrazine, a food pigment with good water solubility, exception absorbance, and sufficient biocompatibility, was identified as an efficacious molecule for tissue clearing enabled by the K-K relation. This strategy enabled us to achieve tissue clearing in highly scattering tissue phantoms, dissected chicken breast tissue, and live mice. The significance of this approach lies in its general applicability to reduce scattering and modulate the RI in any biological or non-biological systems, thus facilitating imaging, light delivery, and telecommunication through a scattering medium.

### Processes for Tissue Imaging

In one aspect, existing methods for tissue imaging require toxic organic solvents having a high refractive index to reduce the refractive index mismatch between scatterers and aqueous background, or to remove scatterers inside biological tissues. In a further aspect, existing methods can only be applied to achieve clearing in fixed tissues from specific organs of interest, for example in post mortem examination or post-surgical histological examination. In some aspects, existing methods involve the replacement of original tissue components with exogenous chemicals, including, but not limited to, replacement of cellular lipids with hydrogels. In a further aspect, existing methods may further involve electrophoresis an excised specimen, exposing the specimen to hydrodynamic pressure, microwave radiation, or ultrasonic vibration.

In still another aspect, existing methods may be able to preserve three-dimensional structures of tissues, but the tissues must still be removed from the body of a subject and, for example, directly contacted with an exogenous component or composition such as, for example, a tissue clearing composition, a surfactant (e.g. a non-ionic surfactant such as a saponin), a buffer, an enzyme, an anticoagulant, a solvent (e.g. acetone), a non-ionic density gradient medium (e.g. a phthalimide), or any combination thereof. In one aspect, existing methods employing such exogenous components as listed herein still require tissue removal from the subject for visualization through microscopy or other means. In a further aspect, the present methods do not require use of some or all of the above-listed components.

By contrast, in one aspect, the disclosed method is based on refractive index modulation of existing tissue components using the K-K relation, thus enabling minimally invasive tissue clearing in live animals. In a still further aspect, the disclosed method can be employed for optical waveguiding in vivo, thus enabling the inspection of deep tissues and organs in live subjects, including tissues and organs that cannot be removed from living subjects. In one aspect, the disclosed method does not require removing the tissue or organ to be imaged from the body of a living subject. In another aspect, the disclosed method does not require performing a surgical procedure on a living subject to access tissue. In still another aspect, the disclosed method does not require use of toxic solvents, fixatives, or the like, in order to visualize tissues and organs.

### Applications of the Disclosed Method

In one aspect, the disclosed method provides a minimally invasive, intravenous approach for deep-tissue visualization and optical imaging both in biomedical research and clinical inspection. In a further aspect, the method can be used to visualize bones, organs, and/or other tissue of interest well below the surface of the skin using light-based inspection methods after creating a transparent spectral window.

In another aspect, the disclosed method enables the creation of a liquid transient fiber after locally injecting the pigment in a solution, allowing inspection of deep locations inside the body and monitoring biomarker concentrations (e.g., sucrose) by detecting the Raman signals thereof.

In still another aspect, the method can be used for fabricating dynamic windows that change their transmission when the absorptivity of one or more of their components is modulated.

In an aspect, the disclosed method can be used to make low-cost, sharp-band optical filters. In still another aspect, the method can be used for long-range communications through clouds for civilian and military uses.

### Optical Clearing in Tissues

In an aspect, light refraction and reflection occur at interfaces when refractive indices change. In another aspect, biological systems such as tissues are inhomogeneous media with different length scales and refractive indices. In still another aspect, reducing the refractive index mismatch between scatterers and background inside tissues can increase light transmission.

In another aspect, the pigments useful in the disclosed methods are minimally toxic, have good water solubility, and are safe for oral administration. In another aspect, the pigments can diffuse into the aqueous phase of biological tissues. In still another aspect, the disclosed method enables a significant increase in optical transmission in otherwise turbid biological tissue. In one aspect, the method can be conducted in live animals, achieving transmission in muscles and allowing clear visualization of deep bones, vessels, and nerves, without the need for invasive surgical procedures. Further in this aspect, complete recovery of the live animals after performing the method is observed.

### Modulation of Refractive Index

In one aspect, the real part (n) and imaginary part (K) of the refractive index of a material are related by the Kramers-Kronig (K-K) relation. In another aspect, in the frequency domain, the Kramers-Kronig relationship can be represented by the following equation:

$$n ( ω ) - 1 = \frac{2}{π} P . V . {\int }_{0}^{∞} \frac{{ω}^{′} κ ( ω )}{{ω}^{′ 2} - {ω}^{2}} d {ω}^{′}$$

In another aspect, the Kramers-Kronig relation can be rewritten in the wavelength domain as follows:

$$Δ n ( λ ) = n ( λ ) - 1 = \frac{2}{π} P . V . {\int }_{0}^{∞} \frac{κ ( λ )}{{λ}^{′} ( 1 - \frac{{λ}^{′ 2}}{{λ}^{2}} )} d {λ}^{′}$$

In still another aspect, by increasing the imaginary part of the refractive index (absorption of the material) the real part of the refractive index will have a nonlinear change in the neighboring wavelength. Further in this aspect, the refractive index in the longer wavelength will increase.

In one aspect, the real part and imaginary part of refractive indices of dye solutions can be measured by any technique known in the art, such as using an ellipsometer. In another aspect, the real part of the refractive index can be modulated by increasing the imaginary part accordingly.

Exemplary uses of the Kramers-Kronig relation as applied to the disclosed system and method are provided in the Examples.

### Optical Clearing Window

In one aspect, an ideal material for modulating optical clearing only absorbs at its absorption peak, leaving other wavelengths open for light transmission. In one aspect, tartrazine solutions of varying concentrations (from 1 mM to 1.0 M) do not absorb at wavelengths above about 600 nm.

### Method for Imaging an Organ or Tissue in a Subject

In one aspect, disclosed herein is a method for imaging an organ or tissue in a subject, the method including at least the steps of (a) administering a composition comprising a compound to the subject, wherein an interaction between the compound and at least one overlying tissue in the subject creates a transparent spectral window in the at least one overlying tissue; and (b) visualizing the organ or tissue through the at least one overlying tissue. In another aspect, the transparent spectral window is in a UV-Visible region of the electromagnetic spectrum, such as, for example, from about 600 nm to about 1000 nm.

In an aspect, the compound can be selected from tartrazine, methyl red, eosin A, brilliant blue FCF, green S, a combination thereof, or a pharmaceutically acceptable salt thereof. In another aspect, the compound is tartrazine.

In one aspect, up to about 2 g of the compound are administered per kg of body weight of the subject. Further in this aspect, about 0.1, 0.25, 0.5, 0.75, 1, 1.25, 1.5, 1.75, or about 2 g of the compound can be administered per kg of body weight of the subject, or a combination of any of the foregoing values, or a range encompassing any of the foregoing values. In an alternative aspect, performing the method results in a local concentration of the compound in the at least one overlying tissue of from about 0.16 M to about 0.62 M, or of about 0.16, 0.20, 0.24, 0.28, 0.32, 0.36, 0.40, 0.44, 0.48, 0.52, 0.54, 0.60, or about 0.62 M, or a combination of any of the foregoing values, or a range encompassing any of the foregoing values.

In any of these aspects, the subject can be a mammal or a bird. In one aspect, the mammal can be a human, rat, mouse, rabbit, guinea pig, hamster, cat, dog, pig, sheep, cow, or horse, or the bird can be a chicken, turkey, duck, parrot, or finch.

In still another aspect, the organ or tissue is visualized in situ in the subject. In one aspect, performing step (a) reduces light scattering between two or more tissue components, the tissue components having different refractive indices. In another aspect, performing step (a) increases light transmittance through the at least one overlying tissue by at least 50-fold compared to light transmittance through the at least one overlying tissue before performing the method, or by at least 10, 20, 30, 40, 50, or 60 fold, or a combination of any of the foregoing values, or a range encompassing any of the foregoing values. In any of these aspects, performing the method can allow visualization of at least one feature in the subject at least 200 μm below the skin surface of the subject.

In one aspect, visualizing can be accomplished using reflectance imaging, fluorescence imaging, laser speckle imaging, two-photon excitation spectroscopy, or a combination thereof. In another aspect, the organ or tissue can be selected from bones, blood vessels, neural tissue, muscle tissue, a tumor, or any combination thereof.

In one aspect, the composition can be administered to the subject by injection, intravenously, subcutaneously, topically, or any combination thereof. In any of these aspects, the compound is non-toxic and, following visualizing, the compound can be excreted by the subject. In one aspect, the compound can be excreted in less than about 10 hours, or in less than about 6 hours, or less than 10, 9, 8, 7, 6, 5, 4, 3, 2, or 1 hour, or a combination of any of the foregoing values, or a range encompassing any of the foregoing values.

### Method for Increasing Transmittance of Light Through a Substrate

In one aspect, disclosed herein is a method for increasing transmittance of light through a substrate, the method including applying a composition including a compound to the substrate, wherein an interaction between the compound and the substrate creates a transparent spectral window in the substrate.

In another aspect, the transparent spectral window is in a UV-Visible region of the electromagnetic spectrum, such as, for example, from about 600 nm to about 1000 nm. In an aspect, the compound can be selected from tartrazine, methyl red, eosin A, brilliant blue FCF, green S, a combination thereof, or a salt thereof. In another aspect, the compound is tartrazine.

In any of these aspects, the substrate can be a living organism, a tissue sample, an optical fiber, a window, or another article having at least two components having different refractive indices. In one aspect, performing the method reduces light scattering between two or more components of the substrate, wherein the two or more components have different refractive indices. In another aspect, performing the method increases light transmittance through the substrate by at least 50-fold compared to light transmittance through the substrate before performing the method, or by at least 10, 20, 30, 40, 50, or 60 fold, or a combination of any of the foregoing values, or a range encompassing any of the foregoing values. In still another aspect, performing the method increases transmittance of light through the substrate to a depth of at least 200 μm below an outer surface of the substrate. In any of these aspects, performing the method results in a concentration of the compound in at least a portion of the substrate of from about 0.16 M to about 0.62 M, or of about 0.16, 0.20, 0.24, 0.28, 0.32, 0.36, 0.40, 0.44, 0.48, 0.52, 0.54, 0.60, or about 0.62 M, or a combination of any of the foregoing values, or a range encompassing any of the foregoing values.

### Method for Imaging an Article

In one aspect, disclosed herein is a method for imaging an article, the method including at least the steps of (a) contacting the article with a composition including a compound, wherein an interaction between the compound and at least one portion of the article creates a transparent spectral window in the at least one portion of the article; and (b) visualizing the article.

In another aspect, the transparent spectral window is in a UV-Visible region of the electromagnetic spectrum, such as, for example, from about 600 nm to about 1000 nm. In an aspect, the compound can be selected from tartrazine, methyl red, eosin A, brilliant blue FCF, green S, a combination thereof, or a salt thereof. In another aspect, the compound is tartrazine.

In any of these aspects, visualizing can be accomplished using reflectance imaging, fluorescence imaging, laser speckle imaging, two-photon excitation spectroscopy, or a combination thereof. In one aspect, the article can be a living organism, a tissue sample, an optical fiber, a window, or another article including at least two components having different refractive indices. In one aspect, performing step (a) can reduce light scattering between two or more components of the article, wherein the two or more components have different refractive indices.

In yet another aspect, performing step (a) increases light transmittance through the at least one portion of the article by at least 50-fold compared to light transmittance through the at least one portion of the article before performing the method, or by at least 10, 20, 30, 40, 50, or 60 fold, or a combination of any of the foregoing values, or a range encompassing any of the foregoing values. In still another aspect, performing the method allows visualization of at least one feature in the article at least 200 μm below an outer surface of the article.

In any of these aspects, performing the method results in a concentration of the compound in at least one portion of the article of from about 0.16 M to about 0.62 M, or of about 0.16, 0.20, 0.24, 0.28, 0.32, 0.36, 0.40, 0.44, 0.48, 0.52, 0.54, 0.60, or about 0.62 M, or a combination of any of the foregoing values, or a range encompassing any of the foregoing values.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by,” “comprising,” “comprises,” “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a dye,” “a medium,” or “a tissue,” includes, but is not limited to, mixtures or combinations of two or more such dyes, media, or tissues, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y.’ The range can also be expressed as an upper limit, e.g. ‘about x, y, z, or less’ and should be interpreted to include the specific ranges of ‘about x,’ ‘about y’, and ‘about z’ as well as the ranges of ‘less than x’, less than y′, and ‘less than z’. Likewise, the phrase ‘about x, y, z, or greater’ should be interpreted to include the specific ranges of ‘about x,’ ‘about y,’ and ‘about z’ as well as the ranges of ‘greater than x,’ greater than y,′ and ‘greater than z.’ In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a dye refers to an amount that is sufficient to achieve the desired improvement in the property modulated by the formulation component, e.g. achieving the desired level of reduction of refractive index differences between or among materials. The specific level in terms of wt % in a composition required as an effective amount will depend upon a variety of factors including the amount and type of organ or other material in which optical clearing is desired, tissue complexity of the organ, identity and solvent of the dye, and the like.

As used herein, the terms “optional” or “optionally” means that the subsequently described event or circumstance can or cannot occur, and that the description includes instances where said event or circumstance occurs and instances where it does not.

As used herein, “overlying tissue” refers to a tissue in the body of a subject positioned over an organ or tissue that is desired to be visualized. The overlying tissue will typically have a different refractive index from the organ or tissue to be visualized and this can create scattering when standard visualization methods are attempted. In one aspect, application of a disclosed compound or composition to the overlying tissue can create a transparent spectral window in the overlying tissue, allowing visualization of structures beneath. In one exemplary aspect, an overlying tissue could be skin, through which muscle is visualized, or an at least one overlying tissue could include both skin and muscle, through which bone, blood vessels, or neural tissue could be visualized. In any of these aspects, the overlying tissue can remain in place while visualization occurs, without the need for surgical intervention, laparoscopy, or the like.

Unless otherwise specified, pressures referred to herein are based on atmospheric pressure (i.e. one atmosphere).

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### ASPECTS

The present disclosure can be described in accordance with the following numbered aspects, which should not be confused with the claims.

Aspect 1. A method for imaging an organ or tissue in a subject, the method comprising:
(a) administering a composition comprising a compound to the subject, wherein an interaction between the compound and at least one overlying tissue in the subject creates a transparent spectral window in the at least one overlying tissue; and
(b) visualizing the organ or tissue through the at least one overlying tissue.

Aspect 2. The method of aspect 1, wherein the transparent spectral window is in a UV-Visible region of the electromagnetic spectrum.

Aspect 3. The method of aspect 1 or 2, wherein the transparent spectral window is from about 600 nm to about 1000 nm.

Aspect 4. The method of any one of aspects 1-3, wherein the compound is tartrazine, methyl red, eosin A, brilliant blue FCF, green S, a combination thereof, or a pharmaceutically acceptable salt thereof.

Aspect 5. The method of aspect 4, wherein the compound is tartrazine or a pharmaceutically acceptable salt thereof.

Aspect 6. The method of any one of aspects 1-5, wherein up to about 2 g of the compound are administered per kg of body weight of the subject.

Aspect 7. The method of any one of aspects 1-6, wherein performing the method results in a local concentration of the compound in the at least one overlying tissue of from about 0.16 M to about 0.62 M.

Aspect 8. The method of any one of aspects 1-7, wherein the subject is a mammal or a bird.

Aspect 9. The method of aspect 8, wherein the mammal is a human, rat, mouse, rabbit, guinea pig, hamster, cat, dog, pig, sheep, cow, or horse.

Aspect 10. The method of aspect 8, wherein the bird is a chicken, turkey, duck, parrot, or finch.

Aspect 11. The method of any one of aspects 1-10, wherein the organ or tissue is visualized in situ in the subject.

Aspect 12. The method of any one of aspects 1-11, wherein performing step (a) reduces light scattering between two or more tissue components, the tissue components having different refractive indices.

Aspect 13. The method of any one of aspects 1-12, wherein performing step (a) increases light transmittance through the at least one overlying tissue by at least 50-fold compared to light transmittance through the at least one overlying tissue before performing the method.

Aspect 14. The method of any one of aspects 1-13, wherein performing the method allows visualization of at least one feature in the subject at least 200 μm below a skin surface of the subject.

Aspect 15. The method of any one of aspects 1-14, wherein visualizing is accomplished using reflectance imaging, fluorescence imaging, laser speckle imaging, two-photon excitation spectroscopy, or a combination thereof.

Aspect 16. The method of any one of aspects 1-15, wherein the organ or tissue comprises bones, blood vessels, neural tissue, muscle tissue, a tumor, or any combination thereof.

Aspect 17. The method of any one of aspects 1-16, wherein the composition is administered to the subject by injection, intravenously, subcutaneously, topically, or any combination thereof.

Aspect 18. The method of any one of aspects 1-17, wherein the compound is non-toxic.

Aspect 19. The method of any one of aspects 1-18, wherein following visualizing, the compound is excreted by the subject.

Aspect 20. The method of any one of aspects 1-19, wherein the compound is excreted in less than about 10 hours.

Aspect 21. The method of aspect 20, wherein the compound is excreted in less than about 6 hours.

Aspect 22. A method for increasing transmittance of light through a substrate, the method comprising applying a composition comprising a compound to the substrate, wherein an interaction between the compound and the substrate creates a transparent spectral window in the substrate.

Aspect 23. The method of aspect 22, wherein the transparent spectral window is in a UV-Visible region of the electromagnetic spectrum.

Aspect 24. The method of aspect 22 or 23, wherein the transparent spectral window is from about 600 nm to about 1000 nm.

Aspect 25. The method of any one of aspects 22-24, wherein the compound is tartrazine, methyl red, eosin A, brilliant blue FCF, green S, a combination thereof, or a salt thereof.

Aspect 26. The method of aspect 25, wherein the compound is tartrazine or a salt thereof.

Aspect 27. The method of any one of aspects 22-26, wherein the substrate comprises a living organism, a tissue sample, an optical fiber, a window, or another article comprising at least two components having different refractive indices.

Aspect 28. The method of any one of aspects 22-27, wherein performing the method reduces light scattering between two or more components of the substrate, wherein the two or more components have different refractive indices.

Aspect 29. The method of any one of aspects 22-28, wherein performing the method increases light transmittance through the substrate by at least 50-fold compared to light transmittance through the substrate before performing the method.

Aspect 30. The method of aspect 29, wherein performing the method increases transmittance of light through the substrate to a depth of at least 200 μm below an outer surface of the substrate.

Aspect 31. The method of any one of aspects 22-30, wherein performing the method results in a concentration of the compound in at least a portion of the substrate of from about 0.16 M to about 0.62 M.

Aspect 32. A method for imaging an article, the method comprising:
(a) contacting the article with a composition comprising a compound, wherein an interaction between the compound and at least one portion of the article creates a transparent spectral window in the at least one portion of the article; and
(b) visualizing the article.

Aspect 33. The method of aspect 32, wherein the transparent spectral window is in a UV-Visible region of the electromagnetic spectrum.

Aspect 34. The method of aspect 32 or 33, wherein the transparent spectral window is from about 600 nm to about 1000 nm.

Aspect 35. The method of any one of aspects 32-34, wherein the compound is tartrazine, methyl red, eosin A, brilliant blue FCF, green S, a combination thereof, or a salt thereof.

Aspect 36. The method of aspect 35, wherein the compound is tartrazine or a salt thereof.

Aspect 37. The method of any one of aspects 32-36, wherein visualizing is accomplished using reflectance imaging, fluorescence imaging, laser speckle imaging, two-photon excitation spectroscopy, or a combination thereof.

Aspect 38. The method of any one of aspects 32-37, wherein the article comprises a living organism, a tissue sample, an optical fiber, a window, or another article comprising at least two components having different refractive indices.

Aspect 39. The method of any one of aspects 32-38, wherein performing step (a) reduces light scattering between two or more components of the article, wherein the two or more components have different refractive indices.

Aspect 40. The method of any one of aspects 32-39, wherein performing step (a) increases light transmittance through the at least one portion of the article by at least 50-fold compared to light transmittance through the at least one portion of the article before performing the method.

Aspect 41. The method of aspect 40, wherein performing the method allows visualization of at least one feature in the article at least 200 μm below an outer surface of the article.

Aspect 42. The method of any one of aspects 32-41, wherein performing the method results in a concentration of the compound in at least one portion of the article of from about 0.16 M to about 0.62 M.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Engineering Principles for Optical Clearing Via the K-K Relation

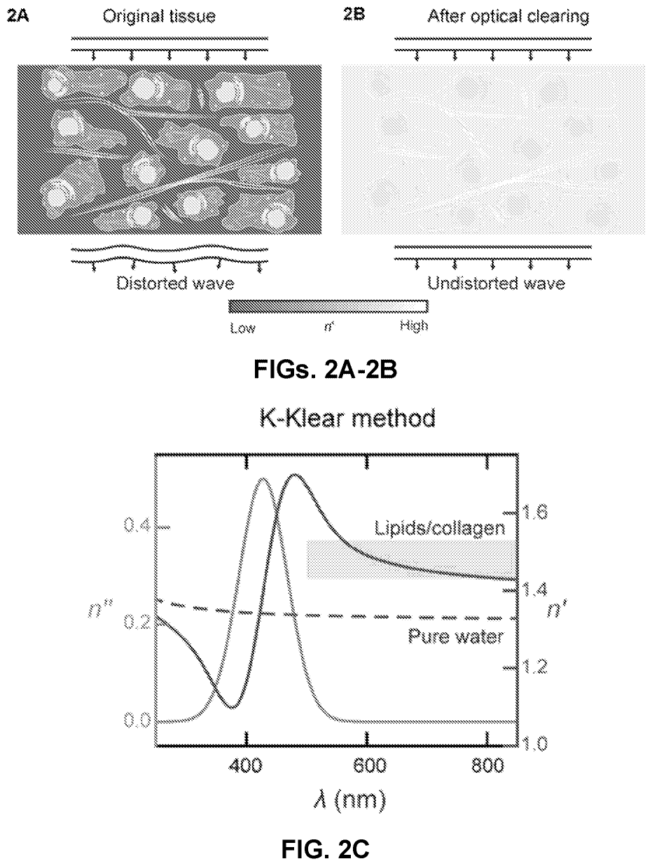

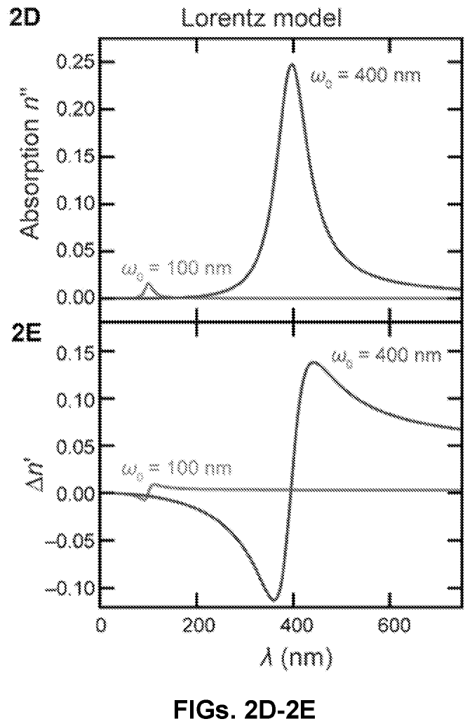

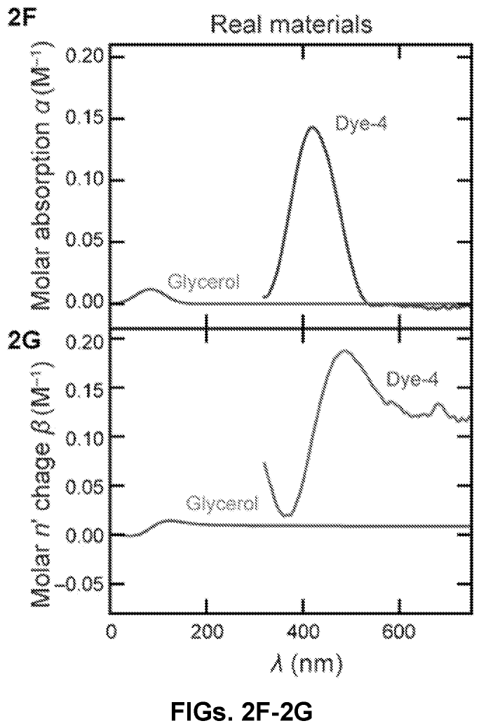

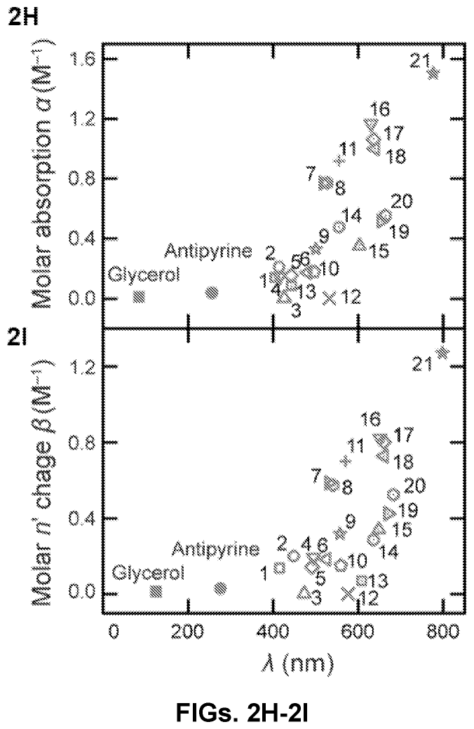

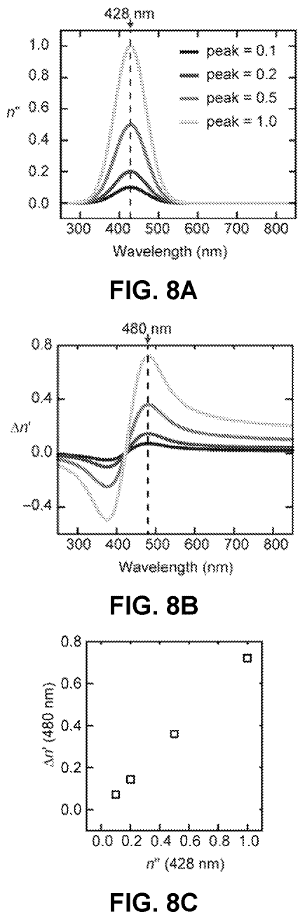

Soft tissues comprise high-n′ components (e.g., lipids) immersed in a low-n′, aqueous environment, thus leading to light scattering (FIG. 2A). Increasing the n′ of the aqueous environment offers an attractive method for tissue clearing by reducing this n′ mismatch and scattering that ensues without removing either water or lipids. Conventional tissue clearing agents cannot efficiently increase the n′ of water when dissolved therein, as evidenced by their limited molar Δn′ coefficient, β (Table 2; see the definition of β in Example 7). In contrast, it is hypothesized that the Kramers-Kronig relation provides a fundamentally different strategy, coined “K-Klear,” to more efficiently increase the n′ of the aqueous component, thus reducing tissue scattering, by introducing intense absorption in the n″ (FIG. 2B). The Kramers-Kronig relation offers a quantitative insight into the way the spectral dependence of n′(λ) can be calculated from its imaginary counterpart n″(λ):

$${n}^{′} ( λ ) = 1 + \frac{2}{π} P . V . {\int }_{0}^{∞} {{n}^{′′} ( λ ) [ {λ}^{′} ( 1 - \frac{{λ}^{′2}}{{λ}^{2}} ) ]}^{- 1} d {λ}^{′} \qquad ( 1 )$$

where λ is the wavelength of light and P. V. denotes the Cauchy principal value of the integral. This equation shows that the spectral location, spectral width, and the strength of the absorption of a dye are all important parameters to control the achievable changes in n′(λ). As a numerical example, FIG. 2C shows that significant changes in the n′ of water (order 0.1) can be achieved in the visible spectrum by dissolving by a stereotypical dye that produces a Gaussian absorption peak centered at 428 nm with a full-width at half-max (FWHM) of 94 nm and a peak absorption coefficient μₐ=4πn″λ−1=0.015 nm⁻¹ (corresponding to a value of n″=0.5). Consequently, the n′ of water increases significantly at >428 nm, reaching that of lipids and collagen fibers (n′=1.43-1.53). Notably, the n′ stays significantly higher than that of pure water (n′=1.33), even at wavelengths where n″ has essentially returned to zero and the absorption is negligible. The achievable changes in n′ are proportional to the increase in n″ (FIGS. 8A-8C), thus suggesting the possibility to quantitatively engineer the n′ at a targeted wavelength by changing the absorption of the medium at a shorter wavelength.

It is argued that K-Klear provides a more efficient strategy for optical clearing since optically absorbing molecules can achieve a higher n′ than conventional RI-matching agents by introducing a stronger resonance. The classical Lorentz model predicts that the dielectric constant of a Lorentz oscillator in the optical frequency range (i.e., 300˜3000 THz) is given by

$${ε}_{r} = 1 + \frac{{ω}_{p}^{2}}{{ω}_{0}^{2} - {ω}^{2} - i γ ω} \qquad ( 2 )$$

where ωₚ is the plasma frequency and given by

$${ω}_{p} = \sqrt{\frac{N {e}^{2}}{{ε}_{0} {m}_{0}}} ,$$

and γ is the damping constant and approximately the full width at half maximum (FWHM) of the resonance peak. Choosing an operating frequency ω in the vicinity of a resonance at ω=ω_{θ}−Δω yields

$${ε}_{r} \approx 1 + \frac{N {e}^{2}}{{ε}_{0} {m}_{0} {ω}_{0}} \cdot \frac{1}{2 Δ ω - i γ} \qquad ( 3 )$$

Eq. (3) reveals two important findings for rationally choosing a clearing agent with an intrinsically high n′ by maximizing its dielectric constant &r. First, the clearing agent should have a low resonance frequency ω_0. Since the operation of tissue clearing is usually in the visible spectrum, this requirement predicts that dyes with absorption peaks in the near-UV (N-UV, 300-400 nm) and blue regions of the spectrum are more efficient clearing agents than those with absorption peaks at shorter wavelengths. Second, the operating frequency should be as close to the resonance as possible to minimize Ao while avoiding significant attenuation due to absorption. Since the Lorentz oscillator absorbs light significantly at the resonance, the optimal operating frequency should be located just below the sharp drop of a narrow resonance at ω≈ω₀−γ to afford sufficient transparency.

The Kramers-Kronig relation and the Lorentz model above also elucidate the reason behind the limited β of conventional tissue clearing agents, such as glycerol (Table 2). For any tissue-clearing agent to exert a Δn′ on water in the visible spectrum when dissolved therein, the Kramers-Kronig relation also causally predicts an absorption peak stronger than that of water in the UV spectrum. However, conventional tissue-clearing agents are chosen with their n′-contributing absorption peak in the short-wavelength, extreme-UV (E-UV) spectrum (usually <150 nm) and their operating frequency located in the visible spectrum, too far away from their resonances. It is thus hypothesized that the limited β of these agents is attributed to a small εᵣ which results from a large ω₀ in to eq. (3). To confirm this hypothesis, two Lorentz oscillators were modeled with their resonances at 100 nm and 400 nm (FIGS. 2D-2E). Specifically, the Lorentz resonator at 100 nm exhibits weak absorption in n″, leading to an inefficient increase in n′ in the visible spectrum. This theoretical prediction is validated experimentally, as evidenced by the small molar absorption coefficient, a, at 84 nm, 0.012 M⁻¹, of a common tissue-clearing agent, glycerol, corresponding to a small β of 0.0129 in the visible spectrum (FIGS. 2F-2G, Table 2). In contrast, the Lorentz resonator in the blue region of the visible spectra is theoretically predicted to be a stronger absorber with a higher peak n″, thus resulting in a more efficient increase in n′ at longer wavelengths (FIGS. 2D-2E). This theoretical prediction also agrees with the α and β spectra of a representative dye, Dye-4 of Table 1, with its peak absorption at 428 nm (FIGS. 2F-2G). Importantly, Dye-4 is 11× more efficient than glycerol in raising the n′ of water at 600 nm on a per molar basis (FIG. 2G). This dependence of RI modulation on resonance frequency is further confirmed by plotting α and β of 23 absorbing molecules vs. their peak absorption from 84 nm to 800 nm, exhibiting stronger absorption and more efficient n′ increase at longer wavelengths (FIGS. 2H-2I, Table 1). These 23 molecules include 21 absorbing molecules in the visible spectrum, along with glycerol and antipyrine, which absorb in the short-wavelength UV spectrum. Notably, antipyrine represents one of the best performing n′-matching agents after screening >1,600 chemicals; however, its much lower B than that of the visible-absorbing molecules confirms the wavelength dependence of the achievable n′ imposed by the Lorentz oscillator model. Remarkably, Dye-21 exhibiting the highest β that exceeds 1.2 M⁻¹ due to its absorption in the near infrared (NIR), which contrasts strongly with those of conventional optical clearing agents (0.001-0.01 M⁻¹) with their absorption in the E-UV region.

TABLE 1
Dye Molecules and Formulas

| | | | | Molecular |
|---|---|---|---|---|
| Dye | | | | weight |
| index | IUPAC name | Chemical structure | Formula | (g/mol) |
| | Glycerol | 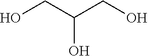 | C₃H₈O₃ | 92.09 |
| | Antipyrine | 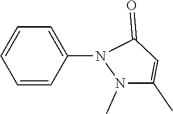 | C₁₁H₁₂N₂O | 188.23 |
| 1 | Trisodium 8-hydroxy- 1,3,6- pyrenetri- sulfonate | 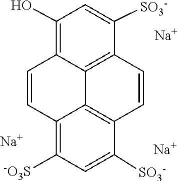 | C₁₆H₇Na₃O₁₀S₃ | 524.39 |
| 2 | Sodium 1H-indene- 1,3(2H)- dione-2-(2- quinolinyl) sulfonate | 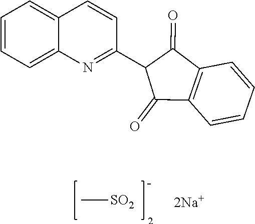 | C₁₈H₉NNa₂O₈S₂ | 477.38 |
| 3 | 1,7-bis(4- hydroxy-3- methoxyphenyl)- (1E,6E)-1,6- Heptadiene- 3,5-dione | 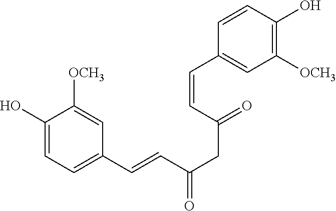 [SMILES: COc1cc(/C=C\C(=O)CC(=O)/C=C/c2ccc(O)c(OC)c2)ccc1O] | C₂₁H₂₀O₆ | 368.38 |
| 4 | Trisodium 5-hydroxy- 1-(4- sulfonato- phenyl)-4- [(E)-(4- sulfonato- phenyl) diazenyl]- 1H-pyrazole-3- carboxylate (a.k.a. tartrazine) | 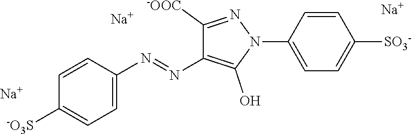 | C₁₆H₉N₄Na₃O₉S₂ | 534.36 |
| 5 | Sodium 4- dimethylamino- azobenzene- 2′-carboxylate (a.k.a. methyl red) | 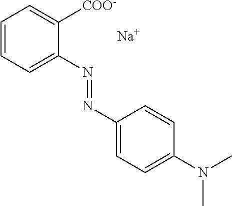 | C₁₅H₁₄N₃NaO₂ | 291.28 |
| 6 | Disodium 6-hydroxy- 5-[2-(4- sulfophenyl) diazenyl]- 2- naphthalene- sulfonate | 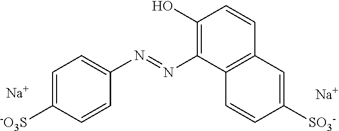 | C₁₆H₁₀N₂Na₂O₇S₂ | 452.36 |
| 7 | Disodium spirofiso- benzofuran- 1(3H),9′- [9H]xanthen]- 3-one- 2′,4′,5′,7′- tetrabromo- 3′,6′-dioxide | 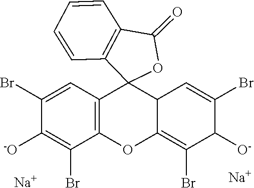 | C₂₀H₆Br₄Na₂O₅ | 691.85 |
| 8 | Disodium spirofiso- benzofuran- 1(3H),9′- [9H]xanthen]- 3-one- 2′,4′,5′,7′- tetraiodo- 3′,6′-dioxide | 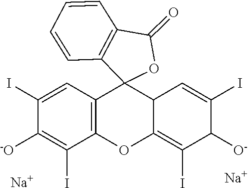 | C₂₀H₆I₄Na₂O₅ | 879.86 |
| 9 | Disodium 3,3′-([1,1′- biphenyl]-4,4′- diyl)bis(4- amino- naphthalene-1- sulfonate) (a.k.a. Congo red) | 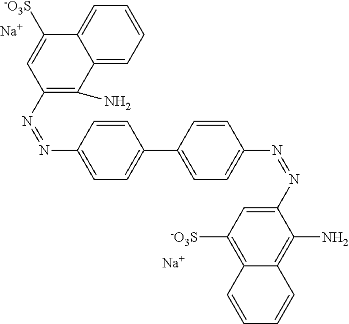 | C₃₂H₂₂N₆O₆S₂Na₂ | 696.66 |
| 10 | Disodium 6-hydroxy- 5-[(2- methoxy-5- methyl-4- sulfophenyl) azo]-2- naphthalene- sulfonate | 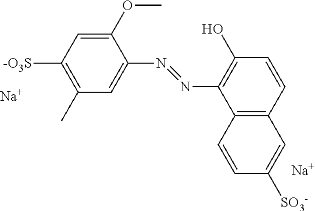 | C₁₈H₁₄N₂Na₂O₈S₂ | 496.42 |
| 11 | 9-(2-Carboxy- phenyl)- 6-(diethyl- amino)-N,N- diethyl-3H- xanthen-3- iminium chloride (a.k.a. Rhodamine B) | 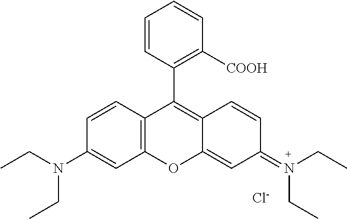 | C₂₈H₃₁ClN₂O₃ | 479.02 |
| 12 | 4-[2-[(2S)- 2-carboxy- 5-(β-D- glucopyrano- syloxy)- 2,3-dihydro-6- hydroxy- 1H-indol-1- yl]ethenyl]-2,3- dihydro- (2S)-2,6- Pyridinedi- carboxylic acid | 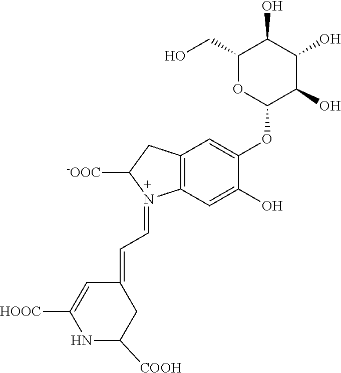 | C₂₄H₂₆N₂O₁₃ | 550.47 |
| 13 | 4,4′-(1,1- dioxido-3H- 2,1-benzo- xathiol-3- ylidene)bis [3-methyl- phenol | 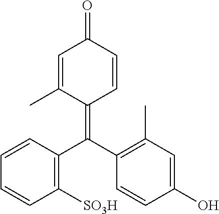 [SMILES: CC1=CC(=O)C=C/C1=C(\c1ccc(O)cc1C)c1ccccc1S(=O)(=O)O] | C₂₁H₁₈O₅S | 382.43 |
| 14 | Sodium benzenemeth- anaminium N-[4-[[4-[(4- ethoxyphenyl) amino] phenyl][4- [ethyl[(3- sulfophenyl) methyl] amino] phenyl] methylene]-2,5- cyclohexa- dien-1- ylidene]- N-ethyl-3- sulfonate | 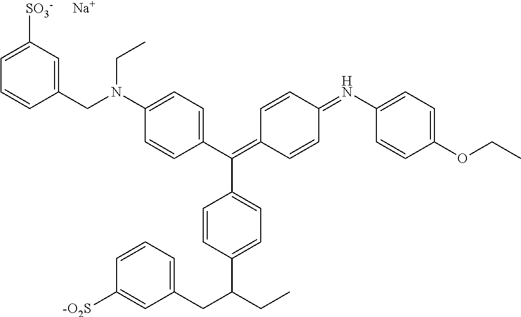 | C₄₅H₄₄N₃NaO₇S₂ | 825.97 |
| 15 | Tetrasodium 4-amino- 5-hydroxy- 3,6-bis[2- [4-[[2- (sulfooxy)ethyl] sulfonyl] phenyl] diazenyl]- 2,7- naphthalene- disulfonate | 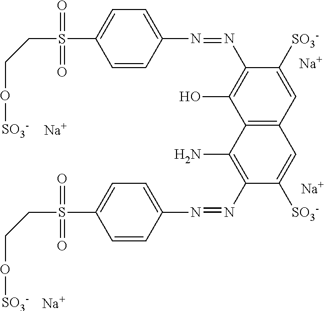 | C₂₆H₂₁N₅Na₄O₁₉S₆ | 991.82 |
| 16 | Disodium benzenemeth- anaminium N-ethyl- N-[4-[[4- [ethyl[(3- sulfophenyl) methyl] amino] phenyl](2- sulfophenyl) methylene]- 2,5-cyclohexa- dien- 1-ylidene]-3- sulfonate | 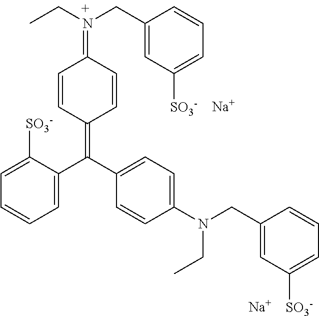 | C₃₇H₃₄Na₂N₂O₉S₃ | 792.85 |
| 17 | Sodium 4-[[4- (dimethylamino) phenyl]-(4- dimethyliminio- cyclohexa- 2,5-dien-1- ylidene) methyl]-3- hydroxy- 7-sulfo- naphthalene-2- sulfonate | 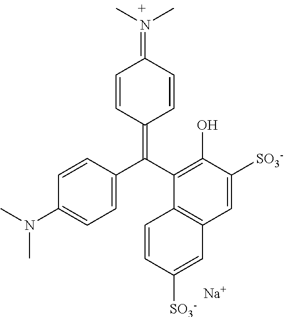 | C₂₇H₂₅N₂NaO₇S₂ | 576.62 |
| 18 | Sodium 2-[[4- (diethylamino) phenyl]- (4- diethyliminio- cyclohexa- 2,5-dien-1- ylidene) methyl]-4- hydroxy- 5-sulfo- benzene- sulfonate | 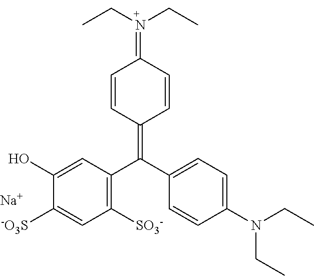 | C₂₇H₃₁N₂NaO₇S₂ | 582.66 |
| 19 | 3,7-bis (diethylamino)- phenoxazin- 5-ium chloride | 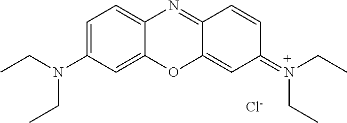 | C₂₀H₂₆ClN₃O | 359.89 |
| 20 | 3,7- bis(dimethyl- amino) phenaza- thionium chloride (a.k.a. methylene blue) | 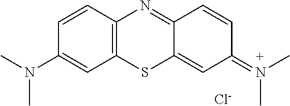 | C₁₆H₁₈ClN₃S | 319.85 |
| 21 | Sodium 4-[2-[2-[2- chloro- 3-[2-[3,3- dimethyl-1-(4- sulfonato- butyl)indol- 1-ium-2- yl]vinyl] cyclohex-2- en-1- ylidene] ethylidene]- 3,3-dimethyl- indolin- 1-yl]butane-1- sulfonate | 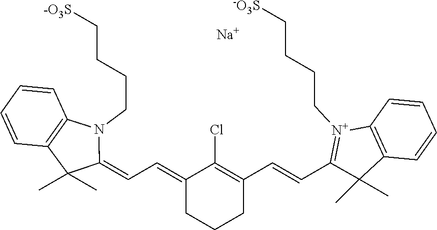 | C₃₈H₄₆ClN₂NaO₆S₂ | 749.35 |

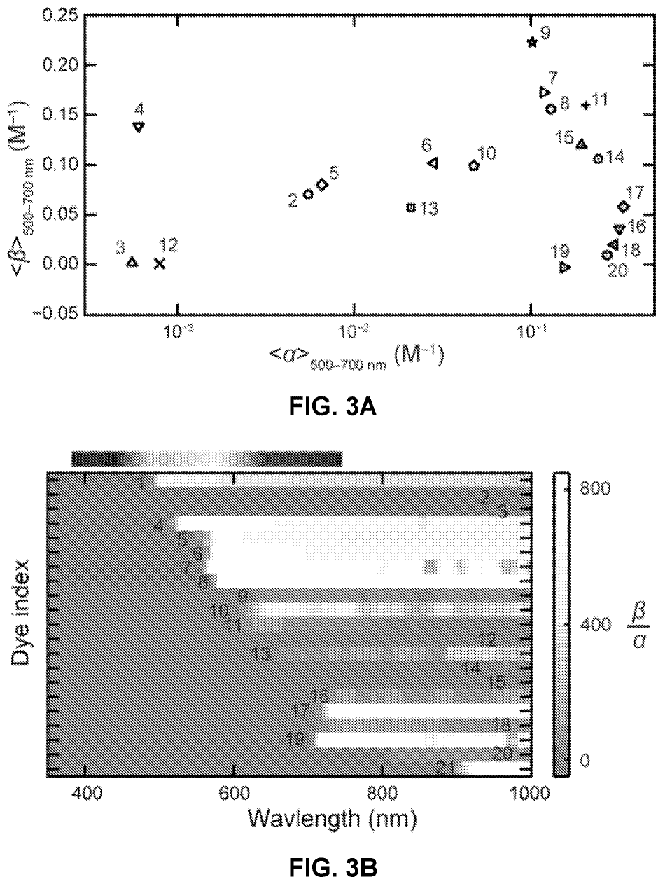

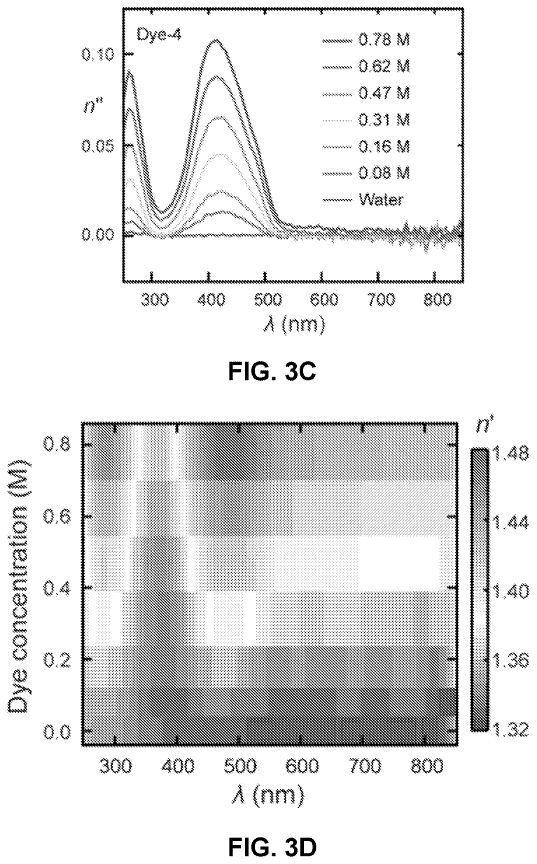

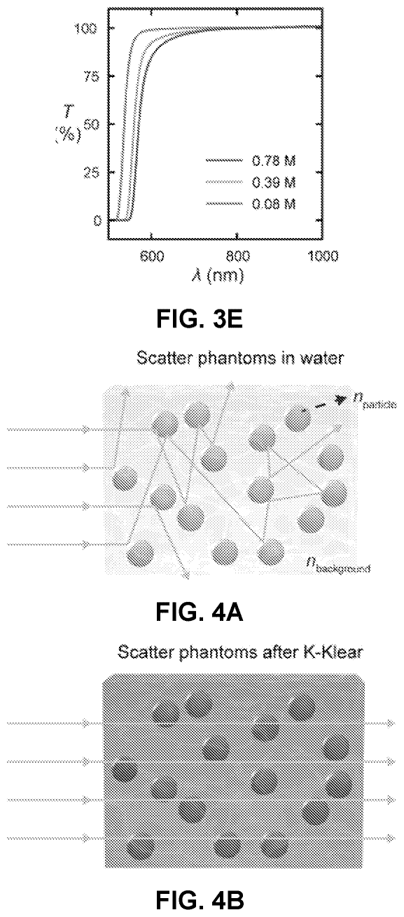

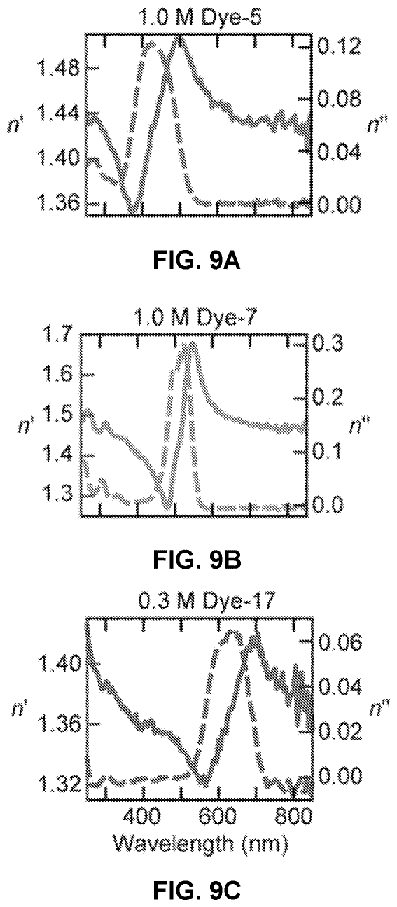

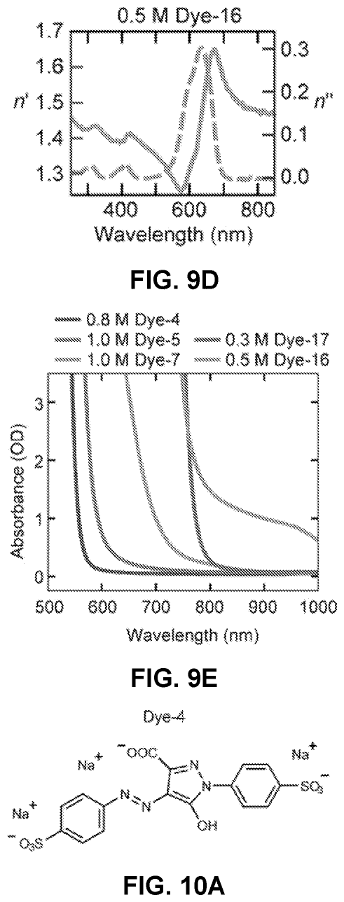

These theoretical and experimental results reveal K-Klear to be a fundamentally different strategy to screen for more efficient tissue-clearing agents that are effective at more feasible and thus biocompatible concentrations. In practice, the agent should display a large absorption coefficient in order to realize the large, required changes in n′. According to the dependence revealed in eq. (3) and FIGS. 2H-2I, the most efficient agent should have its peak absorption at the longest possible wavelength, which is upper-bounded by the wavelength for imaging, to maximize n″ and Δn′. At the same time, the agent should have minimal absorption at the wavelength for imaging, thus calling for a single narrow absorption peak without any additional absorption at longer wavelengths. In addition to these purely optical requirements, the agents also need to display a high solubility and diffusivity in water as well as excellent biocompatibility to facilitate in vivo application. After sampling 21 candidates (FIGS. 3A-3B; FIGS. 9A-9E; and Table 1), one such agent, Dye-4 (Table 1), was identified that satisfies all requirements for effective clearing in the visible spectrum (FIGS. 3C-3E, FIGS. 10A-11C). Specifically, Dye-4 exhibits the highest β while maintaining a low a in the visible spectrum of 500-700 nm (FIG. 3A), as evidenced by a “white window” that appears at the shortest wavelength in the β/α spectrum for all molecules (FIG. 3B). Fourteen other dyes offer similar or even greater potential for clearing at longer wavelengths, with Dye-21 showing the greatest potential for clearing in the NIR spectrum (FIG. 3B).

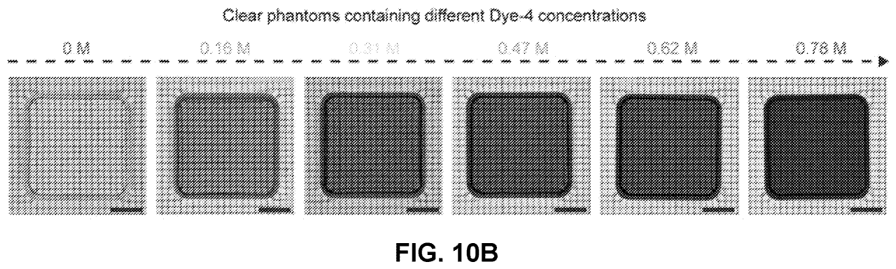

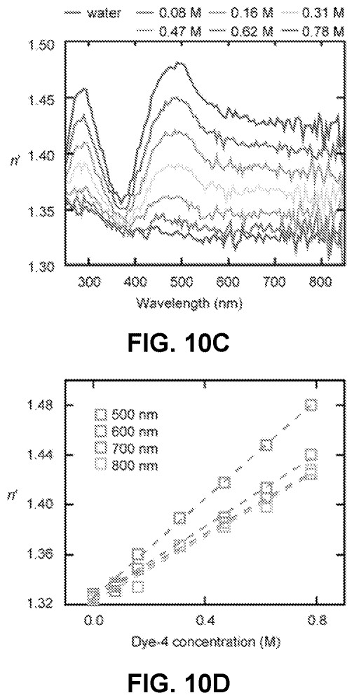

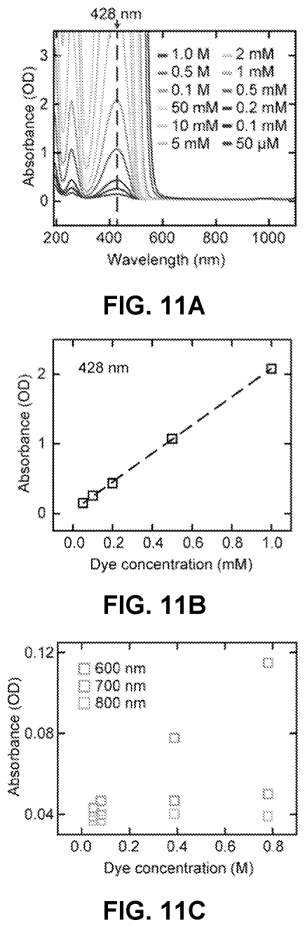

Dye-4, also known as tartrazine, is a yellow, water-soluble mono-azo compound used as a common synthetic pigment in food manufacturing owing to its high absorbance in the blue region of the spectrum. The European Food Safety Authority (EFSA) has concluded that this molecule exhibits no adverse effects or systemic toxicity at doses up to 2 g per kg of body weight. When dissolved in water, Dye-4 exhibits a high absorption coefficient of 2.04±0.02×104 M⁻¹ cm⁻¹ at 428 nm (FIG. 11B). Dye-4 only has a single, narrow absorption peak, and its solution is minimally absorbing above 600 nm (FIG. 3B and FIGS. 11A-11C). The n′ and n″ of aqueous solutions of Dye-4 was measured at different concentrations with ellipsometry (FIGS. 3C-3D). Owing to its strong absorption, the n′ of Dye-4 solution can be changed up to 1.49 at a soluble concentration (FIG. 3D and FIGS. 10A-10D). Remarkably, the measured β of Dye-4 is 2-3 orders of magnitude higher than that of molecules commonly used to raise the n′ of water (Table 2; see the measurements of β in the Example 7).

### Example 2: Demonstrating K-K Optical Clearing in a Scattering Phantom

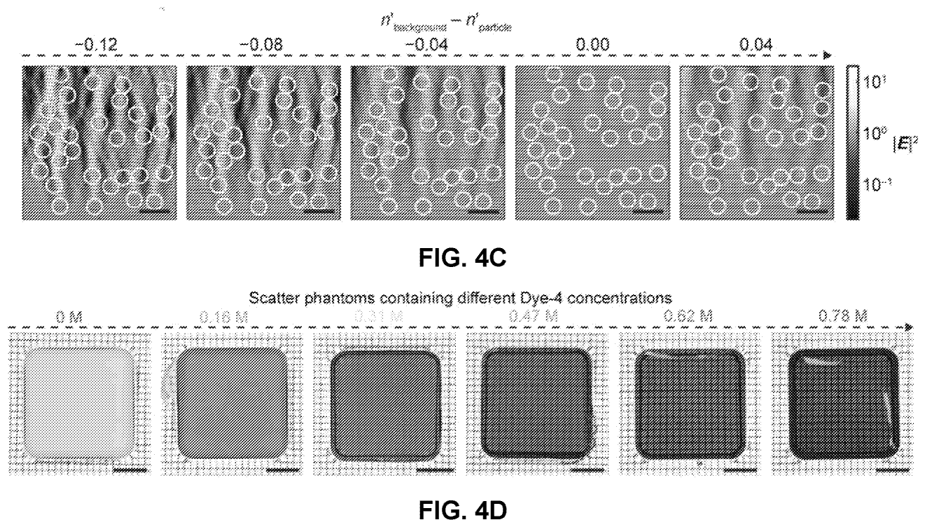

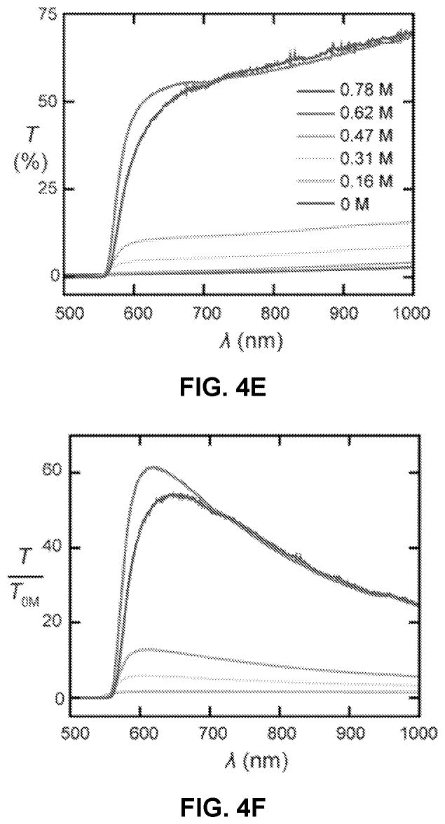

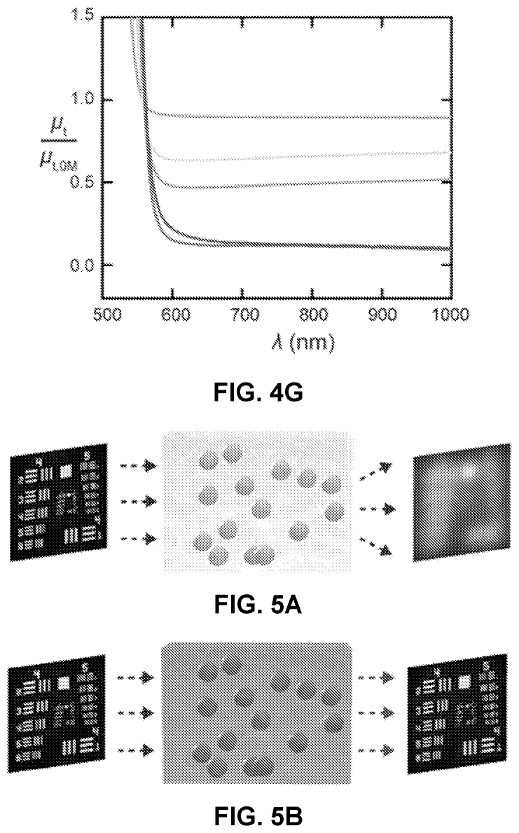

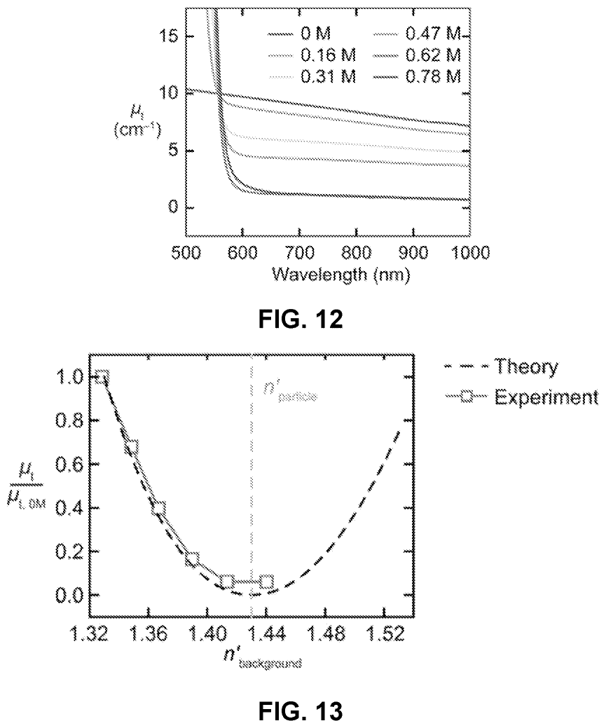

Given the high potential of Dye-4, its optical clearing ability was next demonstrated in a tissue-mimicking scattering phantom (FIGS. 4A-4B). To this end, silica nanospheres were uniformly mixed with an n′ of 1.43 (close to that of lipids and collagen) in an optically transparent hydrogel with a background n′ of 1.33 (the same as water). On the microscopic scale, the interaction between electromagnetic waves and silica nanoparticles can be modeled via finite difference time domain (FDTD) simulations. The silica nanoparticles act as scatterers, thus disturbing the wavefront of the traveling electromagnetic waves inside the hydrogel (FIG. 4C). However, when the n′ of the hydrogel background increases to match that of the scatterers, the incident wave travels without any distortion despite the presence of silica nanoparticles. This prediction was experimentally verified in a scattering phantom with a scattering coefficient of ˜10 cm⁻¹ (FIG. 12), which is on the same order of magnitude as that of muscle. The phantom is then placed on top of a graphing paper showing a regular grid of millimeter-sized squares. Upon increasing the absorbance of this phantom by dissolving Dye-4 into the hydrogel, the opaque phantom gradually turns transparent in the red part of the spectrum (>600 nm) and appears visually similar to the hydrogel without any scatterers, as evidenced by the brightfield photographs (FIG. 4D and FIG. 10B). The effect of optical clearing was quantified by measuring the normal-incidence light transmittance (T) through the phantom as a function of Dye-4 concentration (FIG. 4E). At the optimum clearing wavelength in the red (i.e., >600 nm), a gradual, over-60-fold increase in T was achieved by adding up to 0.62 M of Dye-4 (FIG. 4F). At this concentration, the n′ of the dye-doped hydrogel matches that of the silica nanospheres while producing a negligible n″. Further increases in the concentration of Dye-4 lead to a decrease in maximum transmittance, which is attributed to an overshoot in n′ that causes an increase in scattering, agreeing with the simulation results in FIG. 4C.

The T measurements enable us to extract the scattering coefficient μₛ using the Bouguer-Beer-Lambert law:

$$T = exp ( - {μ}_{t} d ) ≃ exp ( - {μ}_{s} d ) \qquad ( 4 )$$

where the attenuation coefficient μₜ is mainly contributed by μₛ (μₜ=μₐ+μₛ≈μₛ since μₛ>>μₐ) when absorption is negligible beyond 600 nm (FIG. 11C). μₜ was plotted normalized against that of the scattering phantom composed of 0 M Dye-4, μ_{t,0M}, at different concentrations of Dye-4 (FIG. 4G and FIG. 12). A significant reduction in

$$\frac{{μ}_{t}}{{μ}_{t , 0 M}}$$

is found for scattering phantoms with a high concentration of Dye-4 beyond 600 nm, indicating a reduction in light scattering as a result of n′ matching. Moreover,

$$\frac{{μ}_{t}}{{μ}_{t , 0 M}}$$

was plotted as a function of the n′ of the aqueous background of the scattering phantom, which was doped with different dye concentrations (FIG. 13). Importantly, this plot exhibits good agreement with a reported monodisperse scattering model, thus confirming Kramers-Kronig-enabled index engineering as the origin of improved transmittance of the phantoms beyond 600 nm.

### Example 3: Enhancing Imaging Resolution with K-Klear

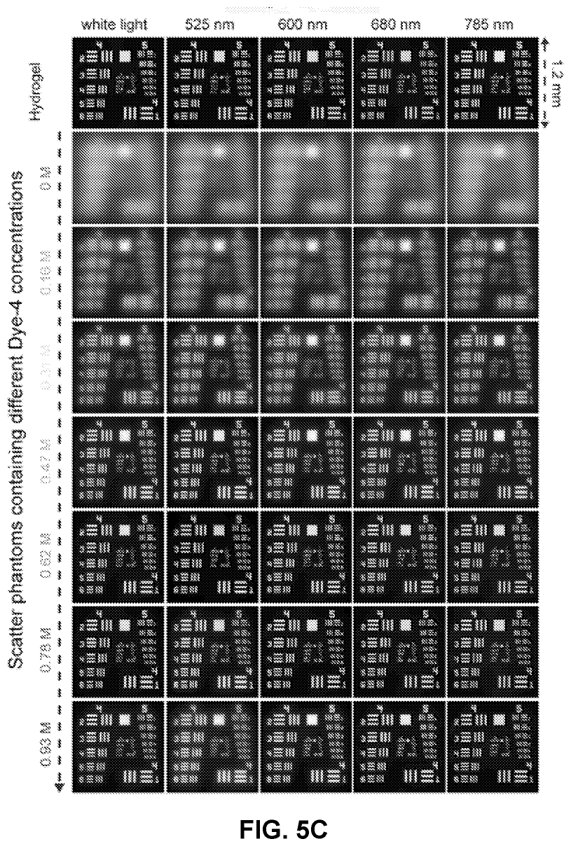

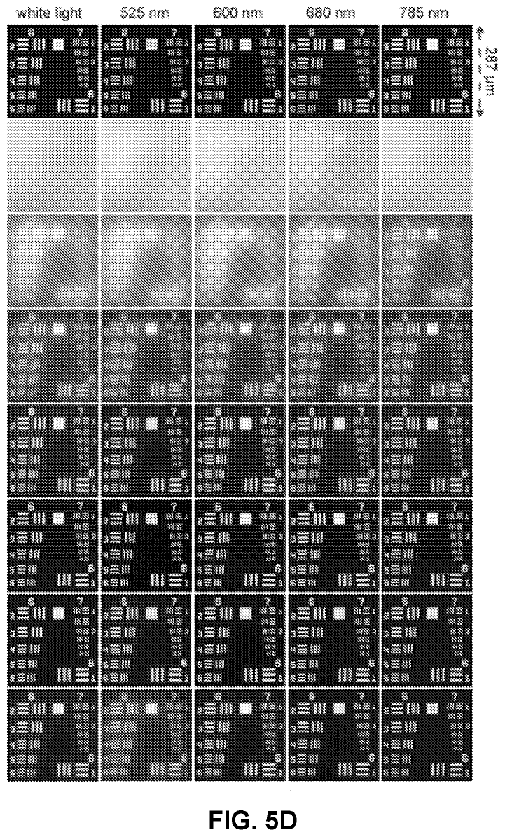

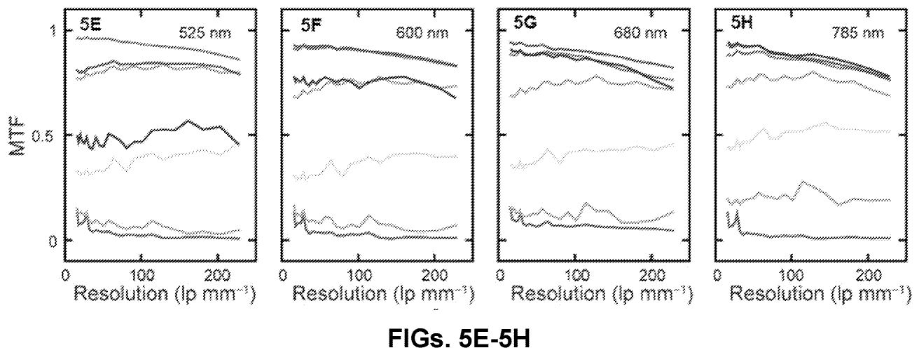

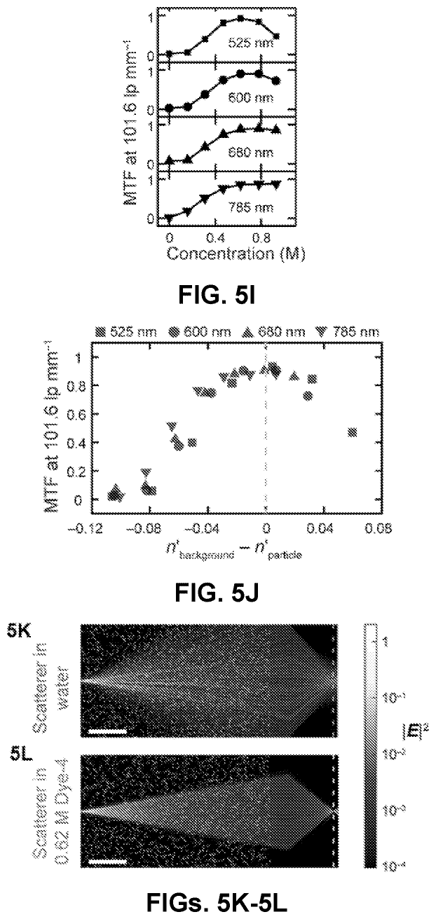

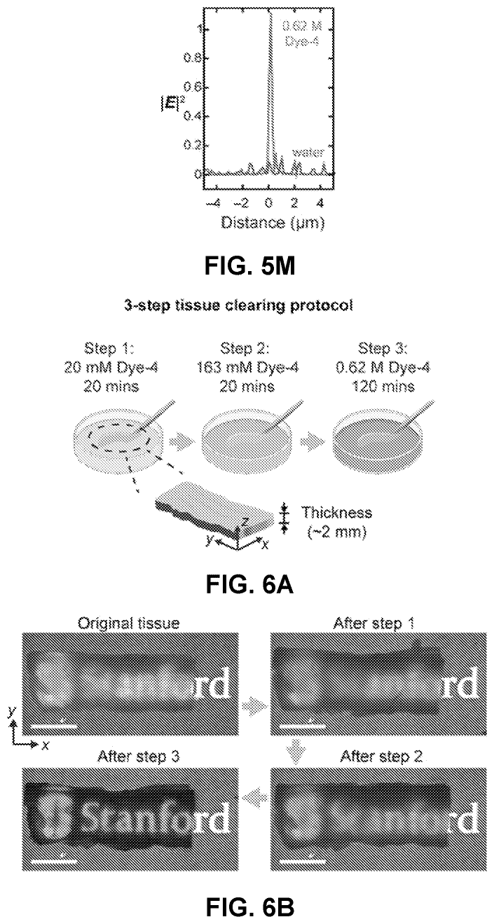

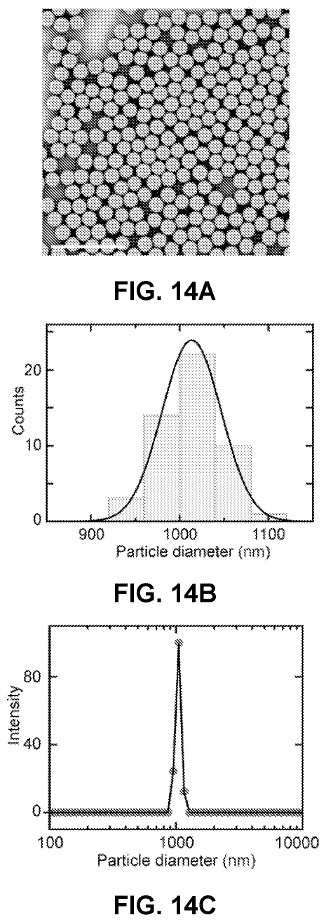

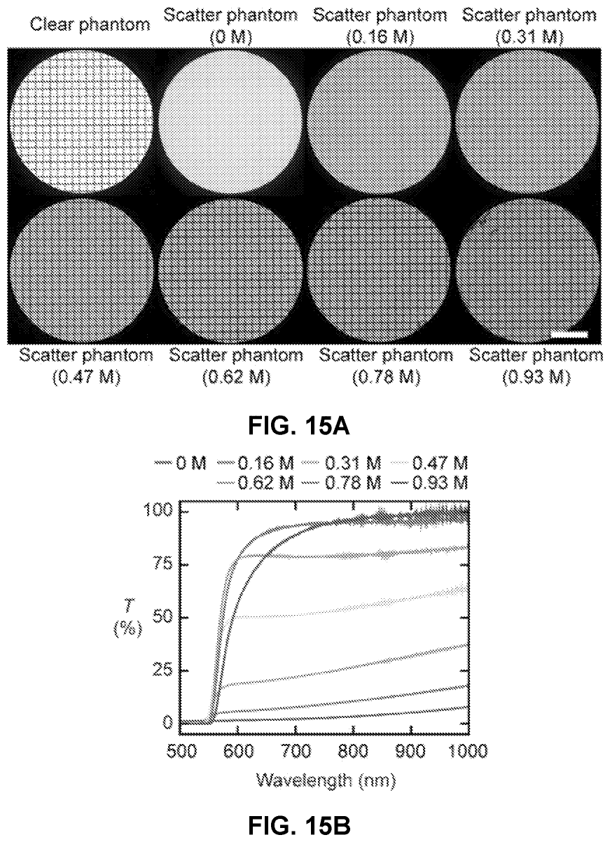

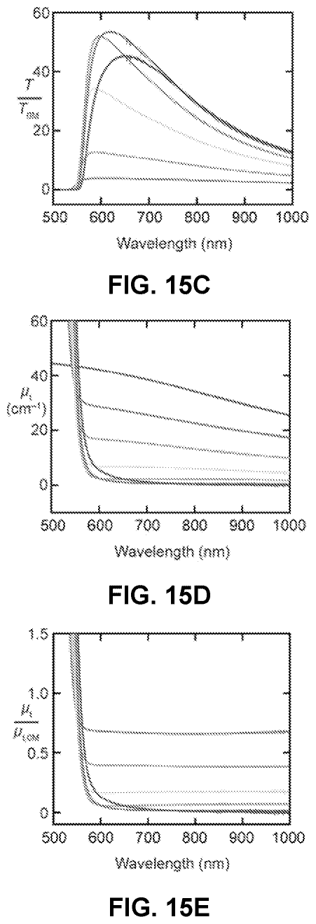

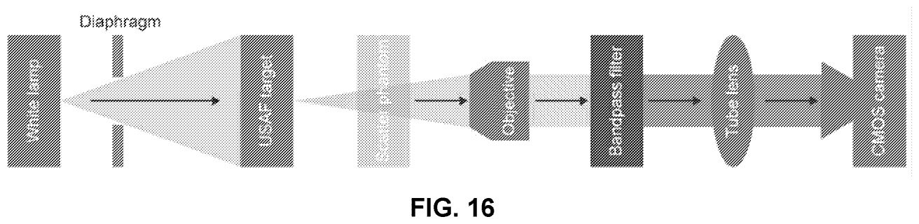

In the following, it is demonstrated that the benefits of optical clearing go beyond enhancing the optical probe depth and also impact the spatial resolution. To this end, the resolvable spatial frequencies of a 1951 US Air Force (USAF) resolution target were quantified when imaging it through 1-mm scattering phantoms with different Dye-4 concentrations (FIGS. 5A-5B; FIGS. 14A-15E; see Example 7 for preparation of the phantom). The scattering phantom is placed between the objective lens and the USAF target, which is back illuminated with a white lamp (FIG. 16). Microscopy images through the clear phantom without any silica particles reveal the smallest features with a line width of 2.19 μm (FIGS. 5C-SD, top row). However, the imaging resolution is greatly impacted in the presence of the scattering phantom (FIGS. 5C-5D, second row). The spatial resolution progressively increases as the dye concentration is increased in the phantom from 0.16 M to 0.62 M (FIGS. 5C-SD, third to last row), and at 0.62 M the 2.19 μm line width can again be resolved. This gradual increase in spatial resolution is quantified through a measured increase in the modulation transfer function (MTF) across all considered spatial frequencies and wavelengths (FIGS. 5E-5H). When the dye concentration is increased above 0.62 M, the MTF exhibits a wavelength-dependent behavior. At the shorter wavelengths, the images show increased blurring, while at the longer wavelengths, the impact on the MTF and spatial resolution reaches a plateau (FIG. 5I).

This behavior is consistent with the spectrally-dependent changes in the RI with dye-doping. At shorter wavelengths, the real index of the aqueous (background) component n′_{background} can overshoot the index of the silica particles. At longer wavelengths, potential overshoots are less pronounced, and the weaker dependence of the index enables us to accurately dial in n′_{background} to achieve clearing across a broad wavelength range (FIG. 3D). Notably, when the MTF at 101.6 lp mm⁻¹ is plotted against the difference between the modulated background real index (n′_{background}) and the particle real index (n′_{particle}), the data at all wavelengths collapse to the same master curve (FIG. 5J). This master curve, along with the clearing effect in the red part of the spectrum (λ>600 nm), validates the K-Klear approach.

These observed experimental data rule out an alternative mechanism that might explain the enhanced image resolution where the scattered photons are preferentially removed by absorbing dyes because of their longer path-length through the scattering medium. To further confirm the mechanism enabled by the Kramers-Kronig relation, numerical, wave-optics simulations have also been carried out to model the interaction of an incident beam with scatter phantoms doped with and without Dye-4 (FIGS. 5K, 5M). The simulations reveal the reduced scattering of 600 nm light waves by Dye-4 due to the Kramers-Kronig relation, rather than the removal of scattered photons by absorption, as the mechanism of the observed clearing effect. Taken together, these data demonstrate the ability to resolve spatial frequencies up to 228.1 lp mm⁻¹ (i.e., line widths down to 2.19 μm) with optimum K-Klear conditions through a 1-mm scattering phantom that optically mimics muscle tissue.

### Example 4: Ex-Vivo K-K Optical Clearing of Biological Tissue

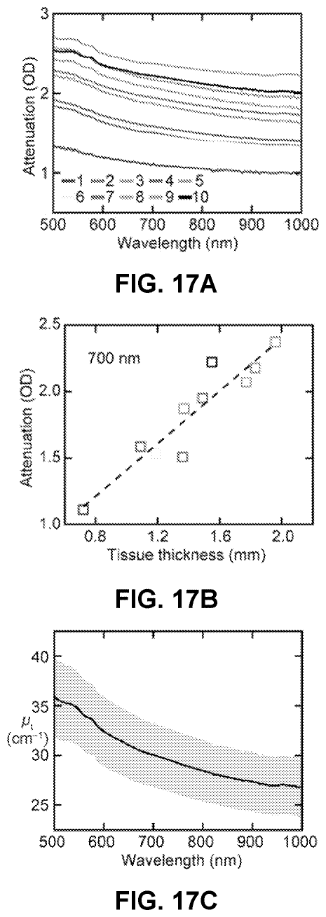

The possibility of using the disclosed clearing approach in real biological tissues is next addressed. To this end, chicken breast was used as a representative, strongly scattering tissue, as evidenced by its us of ca. 30 cm⁻¹ at 600-700 nm as previously reported. The total attenuation coefficient μₜ of chicken breast slices was measured, yielding an average μₜ of 32.5±3.5 cm⁻¹ at 600 nm (FIGS. 17A-17C). Based on this knowledge, it is clear that scattering dominates the attenuation of light, in agreement with previous reports that light attenuation in muscle tissues arises from scattering due to the mismatch between low-n′ (e.g., sarcoplasm and interstitial fluid) and high-n′ components (e.g., myofibrils).

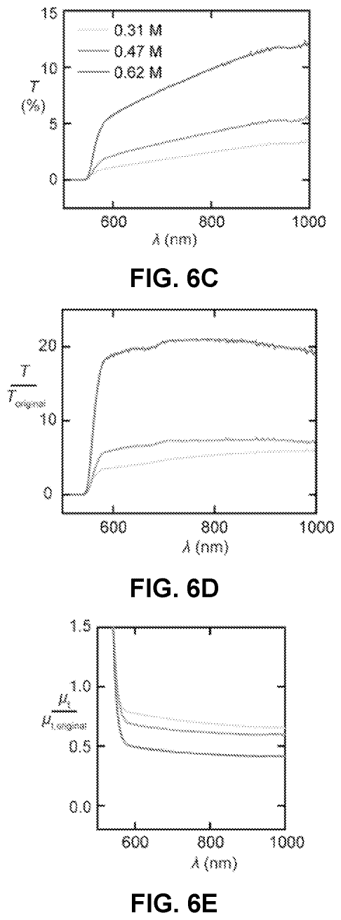

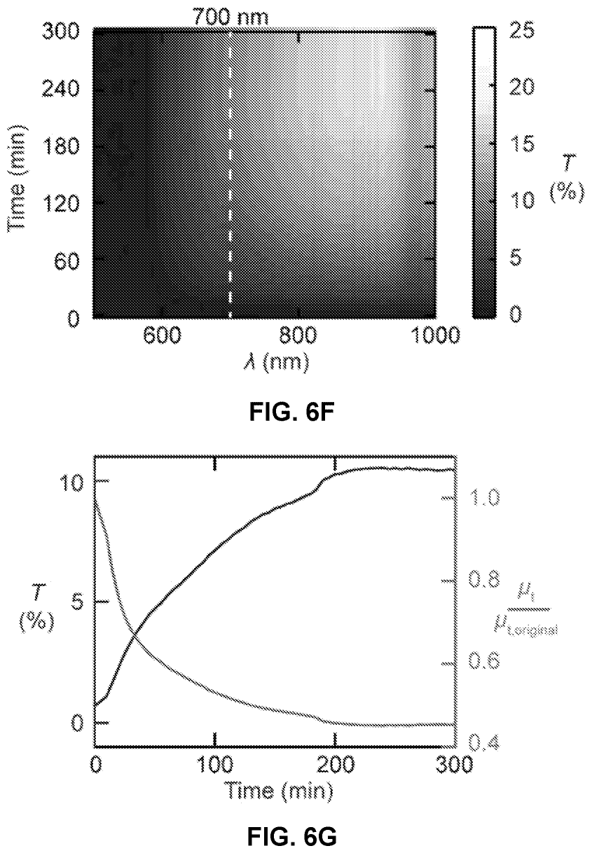

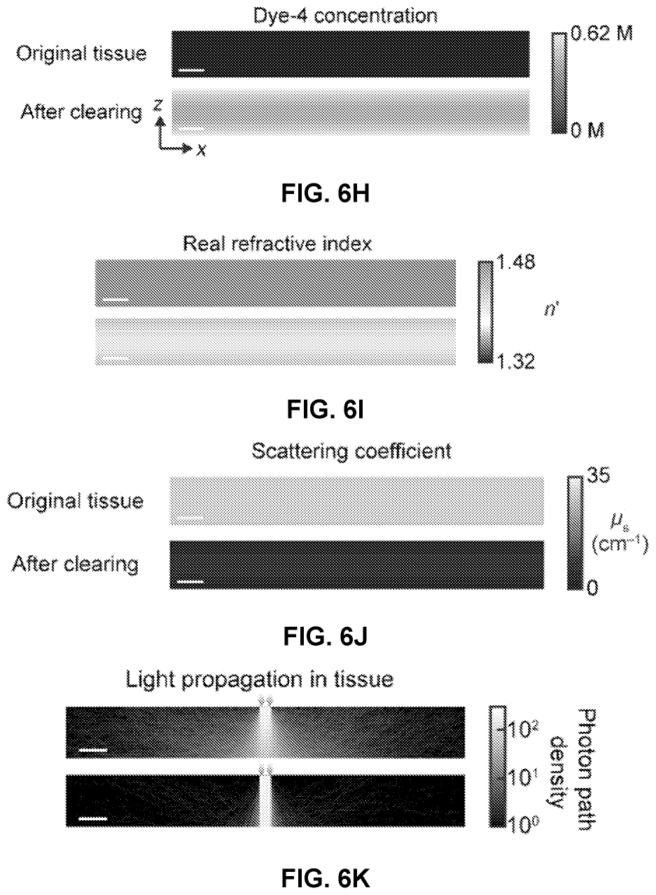

To gradually increase the n′ of the aqueous components in chicken breast, the tissue slices were sequentially immersed in solutions containing an increasing concentration of Dye-4 (FIG. 6A; see Example 7). Brightfield images reveal gradual optical clearing of the tissue with an increasing dye concentration, ultimately enabling the acquisition of a crisp image of the word “Stanford” through a slice thickness of ca. 2 mm, which normally is highly scattering (FIG. 6B). Similar to the demonstration in FIGS. 4A-4G, the red color of the cleared tissue samples was attributed to the creation of an optical clearing window beyond 600 nm (FIGS. 3B, 3E, 4G). Importantly, minimal expansion or shrinkage of the tissue was observed during clearing and the original size and morphology of the sample appears perfectly preserved.

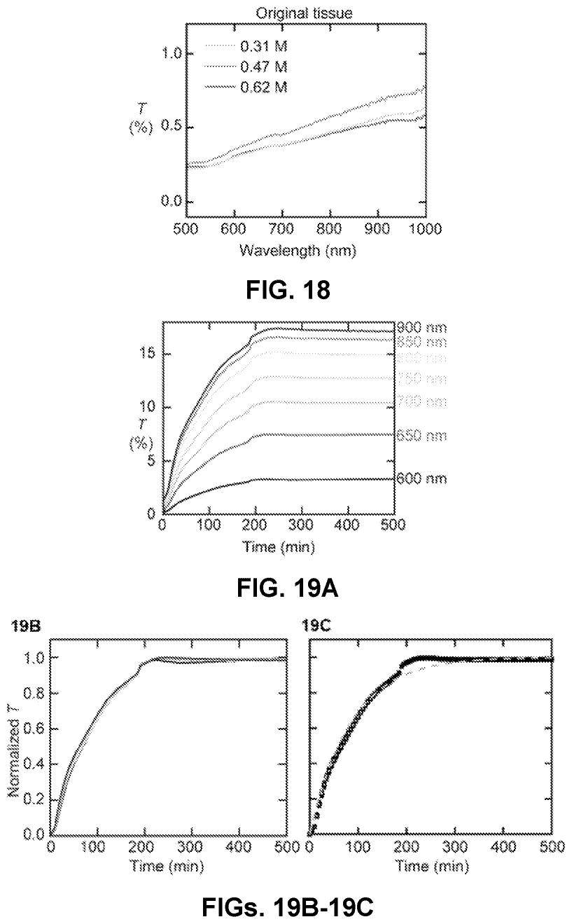

It is hypothesized that the observed optical clearing in the visible can be attributed to increasing the blue absorption of its aqueous components of the chicken tissue. To verify this point, the Dye-4 concentration was varied in the last immersion step and measure T before and after dye immersion (FIGS. 6C and 18). It was found that the enhancement in T at long wavelengths increase monotonically with an increased dye concentration (FIG. 6D). Based on the T measurements, the normalized attenuation coefficient,

$$\frac{{μ}_{t}}{{μ}_{t , original}} ,$$

was calculated for different dye concentrations as a function of wavelength (FIG. 6E). This normalized attenuation coefficient, which is dominated by scattering beyond 600 nm, exhibits a significant reduction down to approximately 40% of the original tissue upon immersion in the 0.62 M Dye-4 solution. This is consistent with the previously observed increase in n′ of the aqueous component in the phantoms at 0.62 M.

In order to predict the required clearing times for tissues of different thickness, the optimum dye immersion time for optical clearing was also evaluated and link it to existing knowledge of dye diffusion. It has been reported that the dynamics of fluid diffusion within the fibrous tissue, such as chicken breast, can be approximated by free diffusion. For this reason, estimates of the diffusion rate of Dye-4 molecules can be made in chicken breast by tracking the temporal evolution of T during dye immersion (FIGS. 6F and 19A-19C). Specifically, the transmission changes for 2-mm-thick chicken breast saturates in approximately 2-3 h (FIG. 6G), suggesting that such immersion times facilitate complete dye penetration into all accessible areas. In contrast, it was found that the required immersion time for sucrose was 24 h to achieve the same n′increase. This difference in required immersion time was attributed to the much higher molar n′ change, β, of Dye-4 than that of sucrose (Table 2). Because it requires a much lower concentration of Dye-4 than sucrose to induce the same Δn′ in an aqueous medium, the Dye-4 solution can be much more dilute than the sucrose solution for achieving the same clearing effect. Using a dilute solution to achieve the same Δn′ and clearing effect benefits from a low viscosity of the clearing agent, thus promoting its diffusion kinetics. As a result, the Dye-4 molecules used in the clearing solution were measured to have a diffusion coefficient of 0.66×10⁻⁶ cm² s⁻¹, in contrast to many conventional high-n′ agents, such as propylene glycol, which has been reported to have a small diffusion coefficient of 0.021×10⁻⁶ cm² s⁻¹ when used as a clearing agent.

TABLE 2
Molar n′ change of Dye-4 in comparison with other high-
n′ chemicals commonly used for tissue clearing

| Chemical | Wavelength (nm) | Molar n′ change β (M⁻¹) |
|---|---|---|
| Dye-4 | 500 | 0.198 ± 0.005 |
| | 600 | 0.143 ± 0.004 |
| | 700 | 0.128 ± 0.004 |
| | 800 | 0.130 ± 0.008 |
| Antipyrine | 600 | 0.037 ± 0.003 |
| Glucose | 589 | 0.0258 |
| Sucrose | 589.6 | 0.0462 |
| Sodium chloride | 589 | 0.00094 |
| Propylene glycol | 589.6 | 0.0084 |
| Glycerol | 589.6 | 0.0129 |

Numerical simulations were next performed to validate the improved optical transparency of chicken breast tissue via the K-Klear method. First, the measured diffusion coefficient of Dye-4 allowed us to simulate the distribution of Dye-4 concentration in a 2-mm muscle sample after immersion in a 0.62 M solution for 2 h (FIG. 6H, FIGS. 19A-19C). Using the ellipsometry data relating the dye concentration with modulated n′ (FIG. 3D, FIGS. 10A-10D), the spatial distribution of the n′ of the aqueous background (FIG. 6I) was then derived. This modulated n′ distribution served as the basis for obtaining the scattering coefficient according to the following equation (FIG. 6J, FIGS. 17A-17C):

$${μ}_{s} ∝ {( {n}_{background}^{′} )}^{3} ( {m}^{2} - 1 ) [ 1 + \frac{2}{{( {m}^{2} + 1 )}^{2}} ] \qquad ( 5 )$$

where m is the n′ratio between the scatterer and the background (i.e., n′_{scatterer}/n′_{background}). Finally, a Monte Carlo simulation was performed for an incident beam on the tissue based on the spatial distribution of scattering coefficients (FIG. 6K). Simulation results of light propagation in the tissue clearly reveal significantly reduced scattering after RI modulation, thus confirming the mechanism of the Kramers-Kronig relation underlying the observed tissue transparency.

### Example 5: In-Vivo K-K Optical Clearing of Live Mice

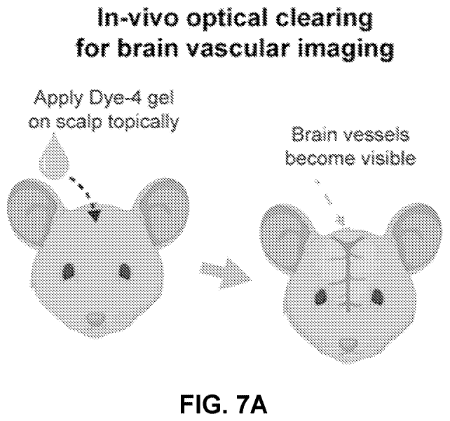

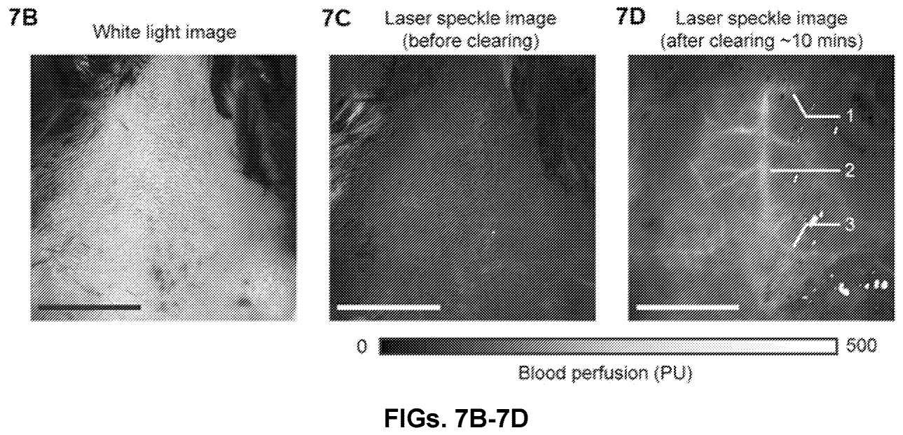

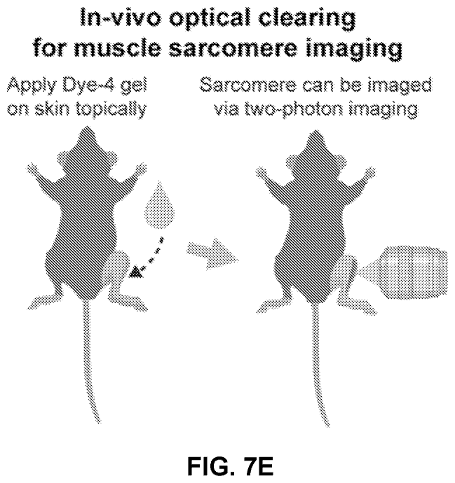

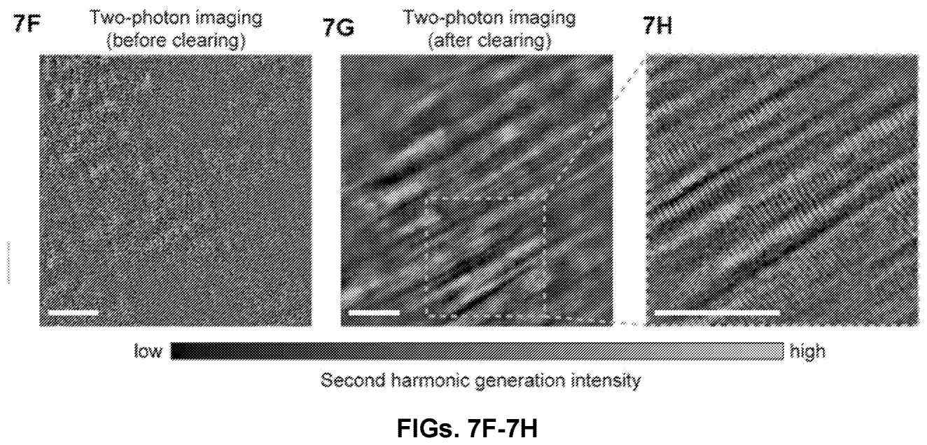

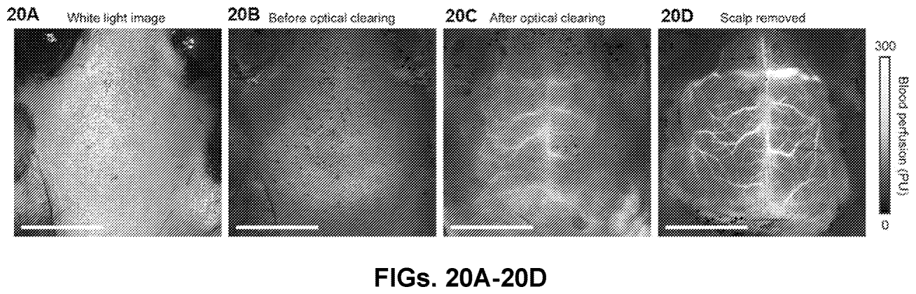

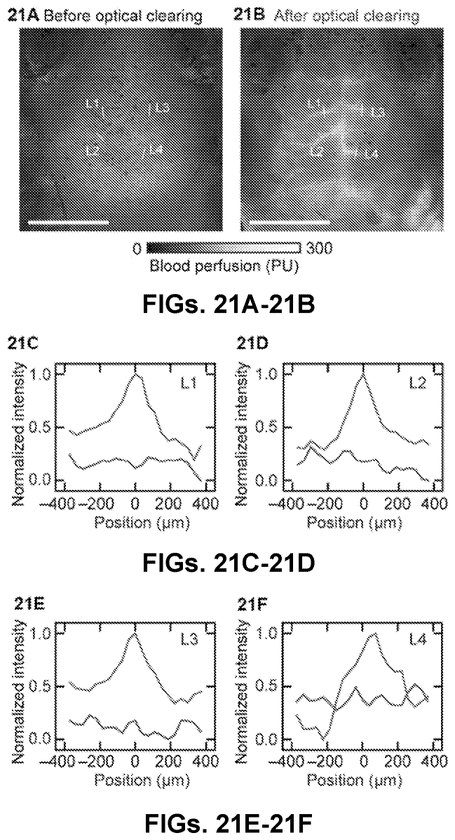

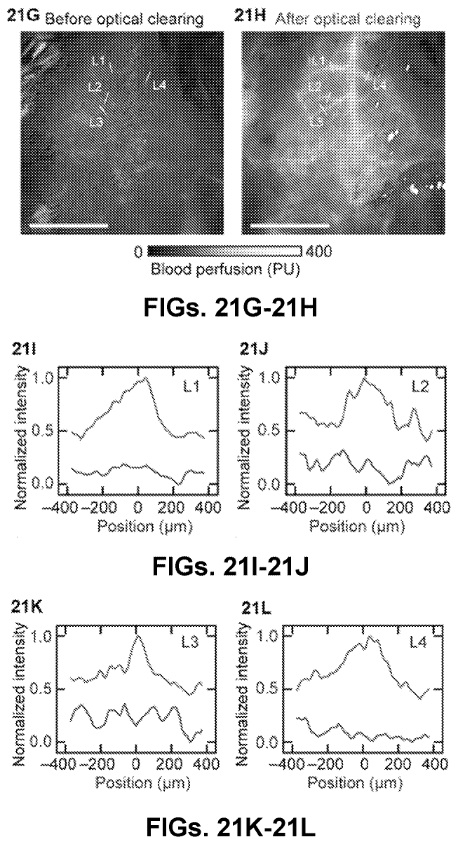

Given the successful clearing of scattering phantoms and chicken breasts using this approach, the possibility to achieve clearing in live animals was next explored. Procedures were developed to circumvent the destructive procedures or the use of toxic chemicals commonly used in ex vivo tissue clearing methods. The first aim was to demonstrate that K-Klear can clear tissue in vivo without any invasive delivery of the agent, Dye-4. It was hypothesized that owing to the small molecular weight and sufficiently large diffusion coefficient of Dye-4 in tissue, it can be delivered topically to optically clear the skin, thus exposing underlying structures that are otherwise obscured by the skin (FIG. 7A). The mouse scalp was chosen to demonstrate this ability, owing to the scalp's opacity as a result of scattering. The opacity of the scalp always requires its removal for fluorescence imaging of neuron activity or laser speckle imaging of cerebral hemodynamics in the mouse brain. To render the scalp transparent, a gel containing 0.62 M Dye-4 was applied topically to the scalp surface and used laser speckle imaging to evaluate the clearing effect. The wavelength of 785 nm used in laser speckle imaging lies in the transparency window of Dye-4 (FIGS. 3B, 3E). The laser speckle image before Dye-4 application only reveals indistinctive features with low signals (FIGS. 7B-7C), likely arising from scattering in the scalp that masks the vascular structures underneath. In striking contrast, after topical application of Dye-4 on the scalp, the image clearly reveals vascular structures representative of cerebral vessels, such as the inferior cerebral vein, superior sagittal sinus, transverse sinus, and their many collateral vessels (FIG. 7D). To confirm that these vessels are indeed located in the brain rather than in the scalp, laser speckle imaging was performed of mouse cerebral vessels after physically removing its scalp, identifying similar structures (FIGS. 20A-20D). Line cross-sectional intensity profiles of two animals revealed that these cerebral vessels could only be observed after the scalp was optically cleared via topical application of Dye-4, while they exhibited indistinct features before clearing (FIGS. 21A-21L). Importantly, it is envisioned that this approach may enable a “scalp window” for surgery-free brain imaging of neural activity and vascular structures through the intact scalp of mice. This surgery-free scalp window will eliminate the challenges of conventional cranial window and skull thinning techniques, such as the significant temperature decrease in the cortex and potential damage to skull bone marrow and meninges.

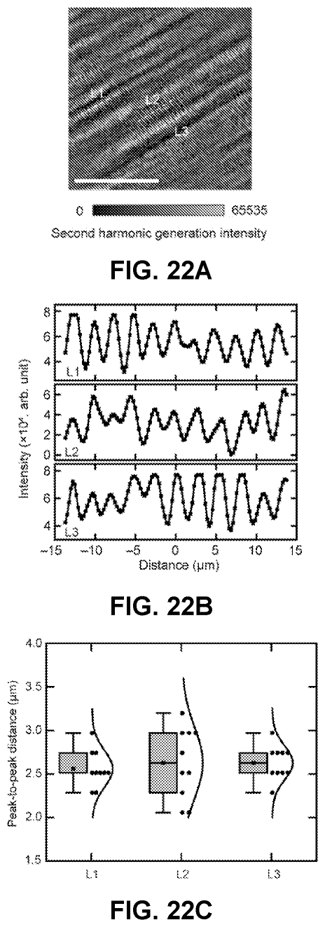

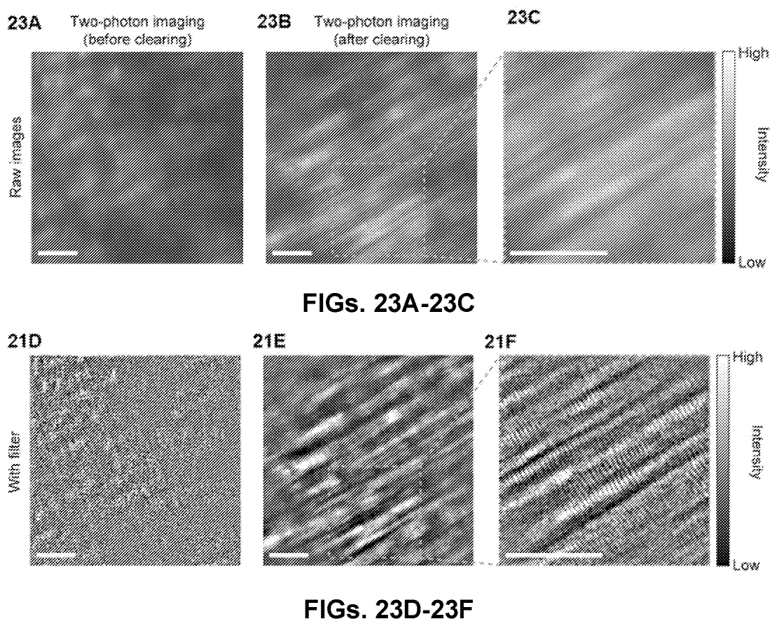

Besides imaging cerebrovascular structures with mesoscopic sizes (e.g., ˜100 μm), it was next sought to demonstrate microscopic imaging in deep tissue with K-Klear. Sarcomeres, basic contractile units of skeletal and cardiac muscles, were chosen as the targets for imaging owing to their central roles in muscle functions and implications in neuromuscular diseases (e.g., spinal muscular atrophy). Existing in vivo sarcomere imaging methods require the invasive implantation of microendoscopes inside the muscle tissue for imaging the second-harmonic generation (SHG) signals from myosin rods in the sarcomere. It was hypothesized that the need for an invasively implanted microendoscope can be alleviated by applying the K-Klear method to reduce the scattering in the skin. To render the skin transparent, a gel containing 0.62 M Dye-4 was applied topically on the mice hindlimb (FIG. 7E). While direct SHG imaging through the intact skin reveals indistinct features at a depth of >200 μm (FIG. 7F), the “skin window” enabled by K-Klear allowed us to identify the periodic structures of sarcomeres at the same depth in vivo without any invasive surgery or implant (FIGS. 7G-7H). Importantly, a characteristic periodicity of 2.60±0.28 μm (FIGS. 22A-23F), which corresponds to the length of sarcomeres, was identified in myofibrils through the skin window (FIG. 7H). This average periodicity value between SHG-generating myosin rods agrees with the reported sarcomere lengths measured via an implanted microendoscope, thus confirming the ability of K-Klear to resolve microscopic features through cleared tissue. Taken together, the above results validate the ability of in vivo optical clearing of the tissue by topically applying the absorbing molecules on the tissue of interest.

### Example 6: Discussion

Despite recent advances in tissue clearing techniques, very few of them can be applied in vivo. This limitation arises from the intrinsic incompatibility of clearing agents used for tissue clearing with biological tissues. The use of toxic organic molecules in existing tissue-clearing approaches, such as tetrahydrofuran and acrylamide, significantly limits their potential for in vivo tissue clearing. In addition, existing n′-matching agents such as glycerol and sucrose exhibit a small molar n′ change, thus necessitating their use at a exceedingly high concentration or even in the pure substance form. As a result, n′ matching requires nearly complete replacement of the original aqueous environment with n′-matching agent, causing significant dehydration and shrinkage of the biological tissue. These limitations thus require a fundamentally different strategy based on first principles to achieve efficacious tissue clearing in vivo.

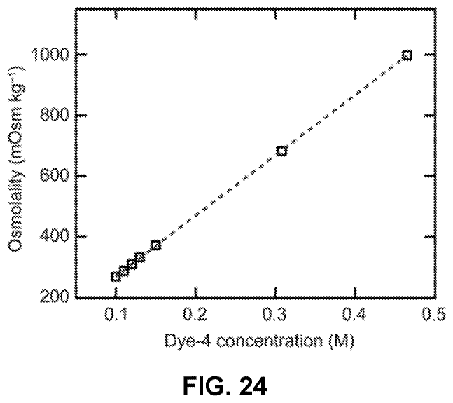

The most significant contribution of the K-Klear method to science lies in a general principle it provides to guide the search for more efficacious tissue clearing agents for improving ex vivo tissue clearing and enabling in vivo tissue clearing. Undesirable tissue deformation ex vivo and the inability to achieve tissue clearing in vivo both result from the small molar n′ change, β, of conventional high-n′ chemicals (Table 2). For example, with a small β of 0.0129 M⁻¹ for glycerol, one must use glycerol at a concentration of 11.6 M, which is almost pure glycerol, to raise the baseline n′ of water to match that of lipids. The use of the high-n′ agents necessarily leads to significant dehydration and deformation of tissue, thus preventing their use for in vivo tissue clearing. The K-Klear method, in contrast, provides both theoretical prediction and experimental validation of high-n′ agents by looking for absorbing molecules with their peak absorption near the clearing spectrum of interest. Remarkably, the reported β values of Dye-4 represent the highest molar n′increase for all n′-matching agents, thus achieving n′ matching at a much lower concentration of the agent. As a result, in vivo tissue clearing can be readily accomplished at an achievable concentration of Dye-4 (FIGS. 7A-7H) that is well below its toxic dose¹⁶ and a tolerable osmolarity when applied topically (FIG. 24). The K-Klear method thus offers an efficient strategy for screening potential efficacious clearing agents based on their absorption spectrum and even designing new clearing agents by rationally introducing chromophore groups into existing molecules. Besides achieving in vivo clearing, this strategy can also be leveraged to significantly expand the library of chemicals for other brain-clearing techniques such as CUBIC.

Dye-4 has been specifically chosen in this study to aid in visualizing the K-Klear effect with the naked eye. However, dyes with an even higher β at longer wavelengths (e.g., Dye-21 in FIG. 2I with 8>1.2 M⁻¹), which is predicted by the Lorentz oscillator model in eq. (3), may open a clearance window in the near-infrared spectrum to facilitate optical imaging therein. Importantly, this very model also sets a fundamental limit on the required concentrations to raise the n′ of water and achieve index matching at a specific wavelength. For example, although one may find a chromophore with strong absorption in the N-UV spectrum to raise the RI across the entire visible region, the required concentration may considerably exceed that needed for Dye-4 due to the intrinsically weaker Lorentz oscillator representing the former. In addition, this model also explains why conventional clearing agents (e.g., glycerol) with optical transparency until the E-UV may never achieve in vivo tissue clearing: their exceedingly small β, which is fundamentally limited by their weak Lorentz oscillator strength, requires prohibitively high concentrations needed to reduce the index mismatch.

Since the Kramers-Kronig relation represents a fundamental physical principle independent of specific chemical and biological contexts, it is envisioned that it can be used in a wide range of applications. Specifically, calcium and voltage imaging of neural activity in the deep mouse brain may be achieved by adapting the K-Klear method in existing widefield, confocal, and two-photon microscopy. In addition, point-of-care glucose sensing may also benefit from this method by creating a transparency window in the skin via topical application of the K-Klear agent, thus allowing for sensitive detection of Raman signals of glucose in underlying capillary vessels. Furthermore, although synthetic dye molecules were delivered to enable in vivo optical clearing in this study, it is envisioned that genetic engineering approaches can be leveraged to express chromoproteins with desirable absorption properties to enable innate optical transparency in mammalian tissues. Besides imaging and sensing, light delivery in vivo can be facilitated by this approach as well. As an example, deep-brain optogenetics may be achieved by modulating the n′ of endogenous brain tissue, thus yielding a “virtual” optical waveguide without any physical implantation of a fiber. The same strategy to guide light delivery may be used to benefit laser-assisted photothermal and photodynamic therapies.

### Example 7: Methods

### Chemicals and Materials

The yellow dye tartrazine (T0388, C₁₆H₉N₄Na₃O₉S₂, ≥85%, molecular weight: 534.36), methyl red sodium salt (114505, C₁₅H₁₄N₃NaO₂, ≥95%, molecular weight: 291.28), and fluorescent dye indocyanine green (ICG, 12633, C₄₃H₄₇N₂NaO₆S₂, ˜85%, molecular weight: 774.96) was purchased from Sigma-Aldrich and used without further purification. E133 brilliant blue FCF, 833 Eosin A, and E142 green S were purchased from FastColors LLP and used without further purification. Dye molecules screened for their K-Klear performance are listed with their full IUPAC names and sources in Table 1.

Hydrochloric acid (HCl, 36.5-38.0 w/w %) and nitric acid (HNO₃, 68.0-70.0 w/w %) were bought from Fisher Scientific and used without further purification. The low melting temperature SeaPlaque™ agarose was bought from Lonza. Silicon dioxide nanospheres (SiO₂, 10-20 nm particle size) were bought from Sigma-Aldrich. Silica nanosphere suspension (1 μm particle size) was purchased from nanoComposix (San Diego, California, USA). The 1× phosphate-buffered saline (PBS) was bought from Gibco. The water used in this work was purified by a Millipore Milli-Q Integral 10 water purification system.

### Ellipsometry Measurement of Solution Refractive Index

A Horiba UVISEL ellipsometer was used to measure the real and imaginary parts of the refractive index of solutions at different concentrations of specific dye molecules. For solution measurement, 5 mL of solution was added to a shallow glass petri-dish (35×10 mm, Corning Falcon). The solution covers the entire bottom of the petri-dish creating a flat, reflective air-liquid interface. The reflected light was measured at an angle of 69.85° and the refractive index of the solution was calculated using a single interface semi-infinite reflection model. For all the measurements, 0.2 s integration time was used for each wavelength.

Measurements of the Molar n′ Change, β

The molar n′ change, β, is defined and measured according to the following quantitative relationship between the n′ and c (i.e., the concentration) of an aqueous solution containing dissolved clearing agents:

$${n}^{′} ( λ ) = {n}_{w}^{′} ( λ ) + β ( λ ) c \qquad ( 6 )$$

where n_{w}′ is the real RI of pure water, c is the molar concentration (M) of the clearing agent, and β is its molar n′ change. Note that n′, n_{w}′, and β all have wavelength dependence. According to eq. (6), by measuring the n′(λ) of the solutions with ellipsometry, one can obtain β(λ) by fitting n′(λ) with c to a linear relationship.

### UV-Vis Transmission Spectroscopy Measurement

A Thermo Fisher Scientific Evolution 350 UV-Vis spectrophotometer was used to measure the transmission and absorption spectra of dye solutions, silica phantoms, and ex-vivo chicken breast tissues. For all the measurements, 0.2 s acquisition time was used for each wavelength.

For transmission measurements of dye solutions, 1 mm optical path quartz cuvettes (high precision, two polished sides, Orient Analytics) were used and the cells were washed with aqua regia (mixture of 36.5-38.0 w/w % HCl and 68.0-70.0 w/w % HNO₃ with a volume ratio of 3:1), fully rinsed with water and dried before use. A reference spectrum was acquired with water alone, thus correcting any loss of light due to intrinsic water absorption and reflection at the air-quartz interfaces.

For transmission measurements of scattering phantoms, the phantom with the mold was placed in the light path of the UV-Vis spectrometer. A clear hydrogel without silica scatterers in the mold was used as the reference to correct for any attenuation due to baseline absorption and reflection at the air-hydrogel interfaces.

For transmission measurements of chicken breast tissue, the tissue sample was first mounted on a microscope slide (precleaned, 12-550-15, Fisher Scientific), which was placed in the light path of the UV-Vis spectrometer to ensure that the entire incident beam is blocked by the tissue. For time-dependent transmission measurement, the tissue was cut to fit into a 1 cm optical path plastic cuvette (acrylic, 14-955-126, Fisher Scientific) while having sufficient area to completely block the incident light. 3 mL of Dye-4 solution was then slowly added to fully immerse the chicken breast tissue, before the cuvette was sealed by parafilm (Bemis Company, Inc., Neenah, Wisconsin, USA) to minimize water evaporation. The temperature of the cuvette was controlled between 25 and 30° C. during measurements.

### Preparation and Imaging of Hydrogel-Silica Scattering Phantoms

The as-purchased silica particles were characterized by an Apreo S LoVac scanning electron microscope (Thermo Fisher Scientific, Waltham, Massachusetts, USA) and NanoBrook Omni particle size and zeta potential analyzer (Brookhaven Instrument, Holtsville, New York, USA) before use. The hydrogel-silica scatter phantom in FIGS. 4A-4G was prepared by first mixing the low melting temperature agarose and silica nanoparticles (10-20 nm) with water or Dye-4 solutions of different concentrations. The final concentration of agarose and silica nanoparticles in the mixture was 6 mg mL⁻¹ and 10 mg mL⁻¹, respectively. The mixture was then heated in an oven at 70° C. for at least 10 min to facilitate the dissolution of agarose. A 1-mL suspension was pipetted into a plastic base mold (15×15×5 mm, Fisher Scientific). The solution was refrigerated at 4° C. for 10 min for solidification. The scatter phantom was then placed on a home-printed transparent pattern illuminated by a white LED panel for imaging.

The hydrogel-silica scatter phantom in FIGS. 5A-5M was prepared by mixing the silica nanoparticle suspension (1 μm, 10 mg mL⁻¹) with low melting temperature agarose and Dye-4 of different amounts. The final concentration of agarose was 6 mg mL⁻¹. In addition, a series of Dye-4 concentrations were achieved according to FIGS. 4A-4G. The mixture was then heated in an oven at 70° C. for at least 10 min to facilitate the dissolution of agarose. A 400-μL suspension was pipetted and injected into the well of a 1-mm silicone isolator (Grace bio-labs, Bend, Oregon, USA) mounted on a coverslip (24×40×0.15 mm, Fisher Scientific). The top of the solution was then sealed by another coverslip (22×22×0.15 mm, Thermo Scientific, Waltham, Massachusetts, USA) and the phantom was left at room temperature for slow solidification. The phantoms were then placed on top of the negative 1951 USAF resolution test target (Thorlabs, Newton, New Jersey, USA) and imaged using an optical microscope (Leica Microsystems, Wetzlar, Germany) with a 10×objective (numerical aperture: 0.25, N Plan Epi BD, Leica Microsystems). White-light illumination was generated by an arc lamp (Newport, Irvine, California, USA). Filters selecting different wavelength ranges were inserted in the front of a scientific CMOS camera (C11440, ORCA-Flash4.0, Hamamatsu Photonics, Japan). Bandpass filters centered at 525 nm and 785 nm were purchased from Semrock (Rochester, New York, USA). Bandpass filters centered at 600 nm and 680 nm were purchased from Thorlabs.

### Ex-Vivo Chicken Breast Preparation, Clearing, and Measurement

Raw boneless skinless chicken breasts were bought from local resources and kept frozen at −20° C. Before experiments, the frozen, raw chicken breasts were allowed to thaw at room temperature for 10 mins. The chicken breasts were then cut into small pieces with ideal thickness in a parallel direction with the fiber orientation and each piece has only one muscle fiber orientation.

The thickness of the tissue was measured by sandwiching the tissue between two coverslips (22×22×0.15 mm, Thermal Scientific) using a digital caliper (Mitutoyo). The transmission spectra of the tissue were measured by attaching the chicken breast tissue on a microscope slide (precleaned, Fisher Scientific). For time-dependent transmission measurement, the tissue sample was sequentially soaked in a 20 mM tartrazine solution for 20 min, a 163 mM tartrazine solution for 20 min, and a 0.62 M tartrazine solution for 2 h. During each step, the sample was placed on an orbital shaker (SHKE4000-7, Thermo Scientific) at 100 rpm and 40° C.

### Vertebrate Animal Subjects

Adult (20 to 30 g) male C57BL/6J mice (for brain vascular imaging, 14-15 weeks old, Jackson Laboratory, Bar Harbor, Maine, USA) and young (10 to 14 g) female BALB/cJ mice (for muscle sarcomere imaging, 3-4 weeks old, Jackson Laboratory) were the animal subjects used in this study. Mice were group-housed on a 12 h: 12 h light: dark cycle in the Stanford University's Veterinary Service Center (VSC) with food and water provided ad libitum as appropriate. All animal experiments conducted were approved by Stanford University's Administrative Panel on Laboratory Animal Care (APLAC) in accordance with Public Health Service Policy on Human Care of Laboratory Animals guidelines and were approved by the Institutional Animal Care and μse Committees of Stanford University.

### In-Vivo Optical Clearing, Reflectance, and Fluorescence Imaging

Before each imaging experiment, the mice were weighed and anesthetized via intraperitoneal (i.p.) injection of a mixture of 16 mg/kg ketamine (Dechra Veterinary Products) and 0.2 mg/kg dexdomitor (Dexmedesed; Dechra Veterinary Products) by insulin syringes with a 30 G needle gauge. The degree of anesthesia was verified via the toe pinch method before the procedure started. To maintain the body temperature and prevent hypothermia of the surgical subject, a homeothermic blanket (Harvard Apparatus) was set to 40° C. and placed underneath the anesthetized mouse. Lubrication eye gel (GenTeal Tears) was applied on both eyes of the mouse to moisturize the eye surface. Hair removal lotion (Nair, Church & Dwight) was used for depilation of the mouse head and both hind limbs. The hair over the mouse head was removed for the in-vivo fluorescence imaging at the mice head. The hair over the mouse hindlimbs was removed for in-vivo reflectance and fluorescence imaging at the hind limbs. Retro-orbital injection of the tartrazine and ICG solutions was conducted using syringes with a 29 G½ needle gauge.

For in vivo optical clearing via topical application of Dye-4, the Dye-4 gel was prepared by mixing the low melting temperature agarose with 0.62 M Dye-4 solution in a 20 mL scintillation vial (FS74504-20, Fisher Scientific) to reach a final agarose concentration of 3-6 mg mL⁻¹. The mixture was then heated in an oven at 70° C. for at least 10 min, followed by refrigeration at 4° C. for 10 min for solidification. For through-scalp brain vascular imaging, after topically applying the Dye-4 gel on the mouse scalp, the mouse head was imaged through a laser speckle imaging system (RWD Life Science Inc., Kent, Delaware, USA) under the sliding mode of imaging with an acquisition time of 5 ms per frame. Every 10 consecutive frames were averaged to improve the signal-to-noise ratio of all images. For transdermal muscle sarcomere imaging, after topically applying the Dye-4 gel on the mouse limb, the sarcomere features were imaged in live mice at a depth of ˜220 μm below the surface of the skin. Specifically, in vivo sarcomere imaging was performed on a laser-scanning microscope (Prairie) equipped with a wavelength-tunable titanium-sapphire laser (Mai Tai, Spectra-Physics). 1040 nm illumination was used and the emission was band-pass filtered (525/50). A 0.3 numerical aperture (NA) objective (Olympus, PlanN) focused illumination onto the mice limb. Images were acquired at 512×512 pixels with 6 us pixel dwell time and averaged over 64 consecutive frames. A band-pass filter selective for 1-5 μm periods was then applied in imageJ, following a previously reported protocol for sarcomere imaging with an implanted microendoscope.

### Numerical Simulation of Light Propagation in Water and Scatter Phantoms

The two-dimensional finite difference time domain (FDTD, Lumerical) method was used to simulate the electric (E) and magnetic (H) vector of light propagating through a domain of 600 μm×1100 μm including no more than two components: silica scatterers and background medium (water or an aqueous solution of Dye-4). A tight-focused Gaussian source with a numerical aperture (NA) of 0.25 was constructed with 400 plane waves using thin-lens approximation, matching the objective used in imaging. For scatter phantoms, silica scatterers with a diameter of 1 μm were randomly placed in the simulation domain reaching a particle density of 0.003 μm 2. The RIs of water and silica scatterers were taken from the literature, and the RI of the 0.62 M Dye-4 solution was taken from ellipsometry measurement (FIGS. 3A-3E and 10A-10D). The simulation time was set as 7500 fs, with a time stability factor of 0.9. Due to the large simulation domain, mesh accuracy was set at 2 with 10 mesh points per wavelength to balance the simulation running time and accuracy, and a conformal variant of 0 was used for mesh setting. The boundary was set as 5 perfectly matching layers and the monitor has a down sampling value of 3.

### Monte Carlo Simulation of Light-Matter Interaction in Tissue

The light propagation in tissue was simulated based on a two-dimensional Monte Carlo method using MATLAB by following a previous publication. The simulation sent photon packets (700 nm) into the tissue with normal incidence, and photon packets were uniformly distributed within a width of 0.4 mm centered on the tissue. The scattering coefficient us of the tissue before clearing was measured from FIGS. 17A-17C (μs>>μa) and that after clearing was calculated based on free diffusion of Dye-4 molecules into the tissue, resulting in n′ and us modulation following Eq. 5. The absorption coefficient μa of the tissue before clearing was taken from the literature and that after clearing was calculated based on the concentration profiles in the tissue and the molar absorbance at 700 nm extracted from FIGS. 11A-11C. The tissue was modeled with a thickness of 1.82 mm and a width of 25 mm to mimic the tissue slices in FIGS. 6A-6K (central 14-mm of the tissue displayed), and a step size of 10 μm was used to discretize and set up the square simulation mesh. Between scattering events, packets were allowed to travel a distance of −ln(RAND)/μs, with the packet's energy decreasing by a factor of exp (−μa×distance). Scattering was considered to be anisotropic, and the scattering angle θ was within a range determined by an anisotropy factor g of 0.93, defined as g=<cos θ>. The distribution of scattering angles was taken from the literature. The refraction and reflection of the packets at the air interface below the tissue were also calculated based on the local n′ according to Snell's Law.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Alieva, N. O.; et al. Diversity and Evolution of Coral Fluorescent Proteins. PLOS One 2008, 3 (7), e2680.
2. Arslan, H.; et al. Optical Penetration Depths and Fluence Distributions in Chicken Breast and Liver Tissues. Opt. Spectrosc. 2019, 127 (4), 763-768.
3. Bashkatov, A. N.; et al. Human Sclera Dynamic Spectra: In-Vitro and in-Vivo Measurements. In Ophthalmic Technologies IX; SPIE, 1999; Vol. 3591, pp 311-319.
4. Bashkatov, A. N.; et al. OPTICAL PROPERTIES OF SKIN, SUBCUTANEOUS, AND MUSCLE TISSUES: A REVIEW. J. Innov. Opt. Health Sci. 2011, 04 (01), 9-38.
5. Bohren, C. F.; et al. Absorption and Scattering of Light by Small Particles; John Wiley & Sons, 2008.
6. Carneiro, I.; et al. A Robust ex Vivo Method to Evaluate the Diffusion Properties of Agents in Biological Tissues. Journal of Biophotonics. 2019, p e201800333.
7. Choi, S. W.; et al. Basic Principles of Hydrogel-Based Tissue Transformation Technologies and Their Applications. Cell 2021, 184 (16), 4115-4136.
8. Chung, K.; et al. Structural and Molecular Interrogation of Intact Biological Systems. Nature 2013, 497 (7449), 332-337.
9. Crank, J.; et al. The Mathematics of Diffusion; Clarendon Press, 1979.
10. Cugurra, A.; et al. Skull and Vertebral Bone Marrow Are Myeloid Cell Reservoirs for the Meninges and CNS Parenchyma. Science 2021, 373 (6553).
11. Deisseroth, K. A. et al; Methods and Compositions for Preparing Biological Specimens for Microscopic Analysis,” U.S. Pat. No. 10,545,075, Jan. 8, 2020.
12. Drew, P. J.; et al. Chronic Optical Access through a Polished and Reinforced Thinned Skull. Nat. Methods 2010, 7 (12), 981-984.
13. Ertürk, A.; et al. Three-Dimensional Imaging of Solvent-Cleared Organs Using 3DISCO. Nat. Protoc. 2012, 7 (11), 1983-1995.
14. Ertürk, A. et al. Three-Dimensional Imaging of the Unsectioned Adult Spinal Cord to Assess Axon Regeneration and Glial Responses after Injury. Nat. Med. 2011, 18 (1), 166-171.
15. Frangioni, J. In Vivo near-Infrared Fluorescence Imaging. Current Opinion in Chemical Biology. 2003, pp 626-634.
16. Frank, J. A.; et al. Next-Generation Interfaces for Studying Neural Function. Nat. Biotechnol. 2019, 37 (9), 1013-1023.
17. Germain, R. N. et al.; Method and Composition for Optical Clearing of Tissues, US 2020/0209117, Jul. 2, 2020.
18. Guo, Z. V.; et al. Flow of Cortical Activity Underlying a Tactile Decision in Mice. Neuron 2014, 81 (1), 179-194.
19. He, F.; et al. Multimodal Mapping of Neural Activity and Cerebral Blood Flow Reveals Long-Lasting Neurovascular Dissociations after Small-Scale Strokes. Sci Adv 2020, 6 (21), eaba1933.
20. Iijima, K.; et al. Optical Clearing of Living Brains with MAGICAL to Extend in Vivo Imaging. iScience. 2021, p 101888.
21. Kalmbach, A. S.; et al. Brain Surface Temperature under a Craniotomy. J. Neurophysiol. 2012, 108 (11), 3138-3146.
22. Kang, J. W.; et al. Direct Observation of Glucose Fingerprint Using in Vivo Raman Spectroscopy. Sci Adv 2020, 6 (4), eaay5206.
23. König, J. 2-Food Colour Additives of Synthetic Origin. In Colour Additives for Foods and Beverages; Scotter, M. J., Ed.; Woodhead Publishing: Oxford, 2015; pp 35-60.
24. Llewellyn, M. E.; et al. Minimally Invasive High-Speed Imaging of Sarcomere Contractile Dynamics in Mice and Humans. Nature 2008, 454 (7205), 784-788.
25. Lucarini, V.; et al. Kramers-Kronig Relations in Optical Materials Research; Springer Science & Business Media, 2005.
26. Malitson, I. H., Interspecimen Comparison of the Refractive Index of Fused Silica. J. Opt. Soc. Am., 1965, 44:1205-1209.
27. Marquez, G.; et al. Anisotropy in the Absorption and Scattering Spectra of Chicken Breast Tissue. Appl. Opt. 1998, 37 (4), 798-804.
28. Ntziachristos, V. Going Deeper than Microscopy: The Optical Imaging Frontier in Biology. Nat. Methods 2010, 7 (8), 603-614.
29. Oliveira, L. M. C.; et al. The Optical Clearing Method: A New Tool for Clinical Practice and Biomedical Engineering; Springer Nature, 2019.
30. Pirdashti, M.; et al. Liquid-Liquid Equilibrium Data, Viscosities, Densities, Conductivities, and Refractive Indexes of Aqueous Mixtures of Poly(ethylene Glycol) with Trisodium Citrate at Different pH. Journal of Chemical & Engineering Data. 2015, pp 3423-3429.
31. Rassier, D. E. Sarcomere Mechanics in Striated Muscles: From Molecules to Sarcomeres to Cells. Am. J. Physiol. Cell Physiol. 2017, 313 (2), C134-C145.
32. Rodríguez-de Marcos, L. V.; et al. Self-Consistent Optical Constants of SiO₂ and Ta₂O₅ Films. Opt. Mater. Express 2016, 6 (11), 3622.
33. Sai, T.; et al. Designing Refractive Index Fluids Using the Kramers-Kronig Relations. Faraday Discuss. 2020, 223 (0), 136-144.
34. Sandell, J. L.; et al. A Review of in-Vivo Optical Properties of Human Tissues and Its Impact on PDT. Journal of Biophotonics. 2011, pp 773-787.
35. Scotter, M. Colour Additives for Foods and Beverages; Elsevier, 2015.
36. Suenson, M.; et al Diffusion of Sucrose, Sodium, and Water in Ventricular Myocardium. Am. J. Physiol. 1974, 227 (5), 1116-1123.
37. Susaki, E. A.; et al. Whole-Brain Imaging with Single-Cell Resolution Using Chemical Cocktails and Computational Analysis. Cell 2014, 157 (3), 726-739.
38. Tainaka, K.; et al. Chemical Landscape for Tissue Clearing Based on Hydrophilic Reagents. Cell Rep. 2018, 24 (8), 2196-2210.e9.
39. Tanzid, M. et al. Imaging through Plasmonic Nanoparticles. Proc. Natl. Acad. Sci. U.S.A 2016, 113 (20), 5558-5563.
40. Tanzid, M.; et al. Absorption-Induced Image Resolution Enhancement in Scattering Media. ACS Photonics 2016, 3 (10), 1787-1793.
41. Tsai, P. S.; et al. Correlations of Neuronal and Microvascular Densities in Murine Cortex Revealed by Direct Counting and Colocalization of Nuclei and Vessels. J. Neurosci. 2009, 29 (46), 14553-14570.
42. Tuchin, V. V. Optical Clearing of Tissues and Blood. 2005. Bellingham, WA: SPIE Publications.
43. Tuchin, V. V. Tissue Optics and Photonics: Biological Tissue Structures. Journal of Biomedical Photonics & Engineering 2015, 1 (1), 3-21.
44. Tuchin, V. V. Tissue Optics and Photonics: Light-Tissue Interaction. Journal of Biomedical Photonics & Engineering. 2015, pp 98-134.
45. Tuchin, V. V.; et al. Light Propagation in Tissues with Controlled Optical Properties. J. Biomed. Opt. 1997, 2 (4), 401-417.
46. Ueda, H. R.; et al. Tissue Clearing and Its Applications in Neuroscience. Nat. Rev. Neurosci. 2020, 21 (2), 61-79.
47. Welch, A. J. et al., eds., Optical-Thermal Response of Laser-Irradiated Tissue; 2011, Springer Netherlands.
48. Wu, X.; et al. Tether-Free Photothermal Deep-Brain Stimulation in Freely Behaving Mice via Wide-Field Illumination in the near-Infrared-II Window. Nat Biomed Eng 2022.
49. Yang, W.; et al. In Vivo Imaging of Neural Activity. Nat. Methods 2017, 14 (4), 349-359.
50. Yu, T.; et al. Physical and Chemical Mechanisms of Tissue Optical Clearing. iScience 2021, 24 (3), 102178.
51. Yun, S. H.; et al. Light in Diagnosis, Therapy and Surgery. Nat Biomed Eng 2017, 1.
52. Zhao, Y.-J.; et al. Skull Optical Clearing Window for in Vivo Imaging of the Mouse Cortex at Synaptic Resolution. Light Sci Appl 2018, 7, 17153.
53. Zhu, D.; et al. Imaging Dermal Blood Flow through the Intact Rat Skin with an Optical Clearing Method. J. Biomed. Opt. 2010, 15 (2), 026008.

1. A method for imaging an organ or tissue in a subject, the method comprising:
(a) administering a composition comprising a compound to the subject, wherein an interaction between the compound and at least one overlying tissue in the subject creates a transparent spectral window in the at least one overlying tissue; and
(b) visualizing the organ or tissue through the at least one overlying tissue.

2. The method of claim 1, wherein the transparent spectral window is in a UV-Visible region of the electromagnetic spectrum.

3. The method of claim 1, wherein the transparent spectral window is from about 600 nm to about 1000 nm.

4. The method of claim 1, wherein the compound is tartrazine, methyl red, eosin A, brilliant blue FCF, green S, a combination thereof, or a pharmaceutically acceptable salt thereof.

5. The method of claim 4, wherein the compound is tartrazine or a pharmaceutically acceptable salt thereof.

6. The method of claim 1, wherein up to about 2 g of the compound are administered per kg of body weight of the subject.

7. The method of claim 1, wherein performing the method results in a local concentration of the compound in the at least one overlying tissue of from about 0.16 M to about 0.62 M.

8. The method of claim 1, wherein the subject is a mammal or a bird.

9. The method of claim 8, wherein the mammal is a human, rat, mouse, rabbit, guinea pig, hamster, cat, dog, pig, sheep, cow, or horse.

10. The method of claim 8, wherein the bird is a chicken, turkey, duck, parrot, or finch.

11. The method of claim 1, wherein the organ or tissue is visualized in situ in the subject.

12. The method of claim 1, wherein performing step (a) reduces light scattering between two or more tissue components, the tissue components having different refractive indices.

13. The method of claim 1, wherein performing step (a) increases light transmittance through the at least one overlying tissue by at least 50-fold compared to light transmittance through the at least one overlying tissue before performing the method.

14. The method of claim 1, wherein performing the method allows visualization of at least one feature in the subject at least 200 μm below a skin surface of the subject.

15. The method of claim 1, wherein visualizing is accomplished Using reflectance imaging, fluorescence imaging, laser speckle imaging, two-photon excitation spectroscopy, or a combination thereof.

16. The method of claim 1, wherein the organ or tissue comprises bones, blood vessels, neural tissue, muscle tissue, a tumor, or any combination thereof.

17. The method of claim 1, wherein the composition is administered to the subject by injection, intravenously, subcutaneously, topically, or any combination thereof.

18. The method of claim 1, wherein the compound is non-toxic.

19. The method of claim 1, wherein following visualizing, the compound is excreted by the subject.

20. The method of claim 1, wherein the compound is excreted in less than about 10 hours.

21.-42. (canceled)
